(19) United States
(12) Patent Application Publication
(10) Pub. No.: US 2025/0114003 A1
(43) Pub. Date: Apr. 10, 2025

(54) SENSORS AND SYSTEMS FOR DETERMINING A PHYSIOLOGICAL PARAMETER USING EXTERNAL PRESSURE

(71) Applicant: QUALCOMM Incorporated, San Diego, CA (US)

(72) Inventors: Emily Kathryn BROOKS, Amherst, NY (US); Hrishikesh Vijaykumar PANCHAWAGH, Cupertino, CA (US); John Keith SCHNEIDER, Williamsville, NY (US); Nicholas BUCHAN, San Jose, CA (US); Vivekananda Parampalli ADIGA, Williamsville, NY (US); Bernard HERRERA SOUKUP, Sunnyvale, CA (US); Ye ZHAN, Buffalo, NY (US); Evan Michael BRELOFF, Kenmore, NY (US); Shounak Uday GORE, Hyderabad, Telangana (IN); Sumit AGRAWAL, Sunnyvale, CA (US)

(21) Appl. No.: 18/480,831

(22) Filed: Oct. 4, 2023

(51) Int. Cl. A61B 5/00; A61B 5/02; A61B 5/0205; A61B 5/021

(52) U.S. Cl. CPC A61B 5/0095; A61B 5/02007; A61B 5/0205; A61B 5/02125; A61B 5/02141; A61B 5/7267; A61B 5/6824; A61B 5/6826; A61B 5/6828; A61B 5/6829; A61B 2560/0228; A61B 2562/0233

(57) ABSTRACT

Sensors and systems for determining a physiological parameter such as blood pressure using external pressure are disclosed. Such sensors and/or systems may be embodied in a wearable user device. The wearable user device may include a cuff configured to apply a pressure to a portion of a user at one or more substantially constant pressure levels; a photoacoustic sensor configured to obtain photoacoustic signals generated from light incident on a blood vessel; and a wearable structure including the cuff and the photoacoustic sensor. Techniques involving the wearable user device may include: obtaining photoacoustic signals from a portion of the user; determining a curve associated with the user, the curve including a plurality of spatial measurements of the blood vessel as a function of a plurality of discrete pressures, the curve enabling determination of a characteristic of the blood vessel; and determining the physiological parameter based at least on the characteristic.

### TECHNICAL FIELD

This disclosure relates generally to devices and systems using biometric sensors, e.g., in conjunction with a cuff.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and/or ambulatory monitoring. Some such devices are, or include, photoacoustic sensors. Although some previously deployed devices can provide acceptable results, improved detection devices and systems would be desirable.

### SUMMARY

The systems, methods and devices of this disclosure each have several aspects, no single one of which is solely responsible for the desirable attributes disclosed herein.

In one aspect of the present disclosure, a wearable user device is disclosed. In some embodiments, the wearable user device may include a cuff configured to apply a pressure to a portion of a user at one or more substantially constant pressure levels; and a photoacoustic sensor configured to obtain photoacoustic signals generated from light incident on a blood vessel of the user, the photoacoustic signals correlated to one or more dimensions of the blood vessel of the user while the pressure is applied to the portion of the user, the one or more dimensions of the blood vessel and the pressure correlating to a characteristic of the blood vessel, the characteristic of the blood vessel enabling determination of a blood pressure of the user; and a wearable structure comprising the cuff and the photoacoustic sensor.

In another aspect of the present disclosure, a method of determining a physiological parameter of a user is disclosed. In some embodiments, the method may include: obtaining photoacoustic signals from a portion of the user using a photoacoustic sensor while a plurality of discrete pressures are applied to the portion of the user at a plurality of corresponding times, the photoacoustic signals generated from light incident on a blood vessel of the user; based on the photoacoustic signals, determining a plurality of dimensions of the blood vessel and a plurality of spatial measurements of the blood vessel corresponding to the plurality of dimensions; determining a curve associated with the user, the curve comprising the plurality of spatial measurements of the blood vessel as a function of the plurality of discrete pressures, the curve enabling determination of a characteristic of the blood vessel at a given pressure; and determining the physiological parameter of the user based at least on the characteristic of the blood vessel.

In another aspect of the present disclosure, an apparatus is disclosed. In some embodiments, the apparatus may include: means for applying a pressure to a portion of a user at a substantially constant pressure level; means for obtaining photoacoustic signals generated from light incident on a blood vessel of the user, the photoacoustic signals correlated to one or more dimensions of the blood vessel of the user while the pressure is applied to the portion of the user, the one or more dimensions of the blood vessel and the pressure correlating to a characteristic of the blood vessel, the characteristic of the blood vessel enabling determination of a blood pressure of the user; and wearable means comprising the means for applying the pressure to the portion of the user and the means for obtaining the photoacoustic signals.

In another aspect of the present disclosure, a non-transitory computer-readable apparatus is disclosed. In some embodiments, the non-transitory computer-readable apparatus may include a storage medium, the storage medium comprising a plurality of instructions configured to, when executed by one or more processors, cause an apparatus to: obtain photoacoustic signals from a portion of a user using a photoacoustic sensor while a plurality of discrete pressures are applied to the portion of the user at a plurality of corresponding times, the photoacoustic signals generated from light incident on a blood vessel of the user; based on the photoacoustic signals, determine a plurality of dimensions of the blood vessel and a plurality of spatial measurements of the blood vessel corresponding to the plurality of dimensions; determine a curve associated with the user, the curve comprising the plurality of spatial measurements of the blood vessel as a function of the plurality of discrete pressures, the curve enabling determination of a characteristic of the blood vessel at a given pressure; and determine a physiological parameter of the user based at least on the characteristic of the blood vessel.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 shows an example of a blood pressure monitoring device based on photoacoustic plethysmography, which may be referred to herein as PAPG.

FIG. 2 is a block diagram that shows example components of a sensor apparatus according to some disclosed implementations.

FIG. 3 shows examples of heart rate waveform (HRW) features that may be extracted according to some implementations.

FIG. 4A shows an example monitoring device designed to be worn around a wrist according to some implementations.

FIG. 4B shows an example monitoring device designed to be worn on a finger according to some implementations.

FIG. 4C shows an example monitoring device designed to reside on an earbud according to some implementations.

FIGS. 5A-5D are diagrams of graphs showing examples of externally applied pressures (e.g., with a cuff) over time according to some implementations.

FIG. 6 depicts ultrasound images of a blood vessel at different levels of pressure.

FIG. 7 depicts dimensions and spatial measurements of a blood vessel experiencing a constant external pressure over time.

FIG. 8 shows an example of an apparatus that is configured to perform a receiver-side beamforming process.

FIGS. 9A and 9B depict beamformed photoacoustic images based on an ultrasonic image of a blood vessel, according to some implementations.

FIG. 10 is a graph of an example curve of arterial spatial measurement as a function of applied external pressure, according to some implementations.

FIGS. 10A and 10B illustrates examples of curves of arterial spatial measurements corresponding to external pressures applied during diastole and systole, according to some implementations.

FIG. 11 is a graph of an example set of data points of arterial dimensions as a function of applied external pressure, according to some implementations.

FIG. 12 is a graph of an example set of data points of arterial cross-sectional areas as a function of applied external pressure, according to some implementations.

FIG. 13 is a flow diagram of a method for determining a physiological parameter of a user, according to some disclosed implementations.

FIG. 14 is a flow diagram of another method for determining a physiological parameter of a user, according to some disclosed implementations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications or monitoring of other physiological parameters. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, chest bands, anklets, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

There is a strong need for accurate, non-invasive, continuous monitoring wearable devices for both clinical and consumer applications, e.g., for measuring physiological parameters such as blood pressure of a user. In particular, non-invasive monitoring of blood pressure is desirable. Continuous blood pressure monitoring opens avenues for efficient and effective diagnosis and treatment of cardiovascular conditions (e.g., hypertension), cardiovascular event detection, and stress monitoring. It would also allow daily spot checks of cardiovascular conditions including blood pressure, as well as overnight sleep monitoring. Positive user experience during overnight sleep monitoring is desirable. For example, there should be minimal discomfort to the user during operation of the wearable device, including during sleep.

Sensing mechanisms that allow collection of biometrics and measurement of physiological characteristics such as pulse wave velocity (PWV) of a blood vessel, arterial compliance, and arterial measurements such as diameter, cross-sectional area, volume, and/or distension, could be a step in that direction. PWV and compliance are relevant characteristics that are a function of the arterial wall stiffness and tension, blood density, body posture, blood pressure, and more. It would thus be valuable for blood pressure estimation to obtain such characteristics with accuracy and convenience.

In particular, compliance can be determined for each individual user and provide information that contributes to accurate estimation of blood pressure of the user. In certain embodiments disclosed herein, photoacoustic signals may be recorded at different pressures applied to the user. For example, a cuff worn by the user can be inflated to different pressure levels, and analysis of signals obtained at these different pressure levels can provide compliance information for the user. Photoacoustic signals offer a unique advantage of enabling measurements of physiological parameters such as dimensions and/or distension of the artery. Measurements of dimensions such as diameter or semi-axes can enable blood pressure estimation in combination with information on arterial stiffness (e.g., compliance). These parameters along with other known information can be used to derive and estimate the blood pressure of the user (in relation to the artery the photoacoustic measurements are taken from).

More specifically, a compliance curve can be determined based on spatial parameters such as arterial volume or cross-sectional area at different pressure levels. Compliance can be defined as a difference of the spatial parameter with respect to a difference in pressure level. In other words, a slope of the compliance curve can yield compliance at a given pressure level. Depending on need, the photoacoustic signals may also be obtained for an initial calibration for age or individual physiology, and intermittent recalibration for drift or positioning of the wearable device. Additional features and information can aid in estimating or predicting the blood pressure of the user, e.g., multiple cuff pressures applied subsequent to obtaining the compliance information, zero-pressure arterial dimension (e.g., diameter of blood vessel at zero applied external pressure extrapolated from measured data), or heart rate waveform (HRW) features. This information can lend itself to providing useful context or an alternate approach for estimating blood pressure.

Further, in some implementations, machine learning can be used to train a machine learning or artificial intelligence model that can predict a physiological parameter of the user (e.g., blood pressure). In addition, based on any discrepancies between the sensor-based estimation and the model-generated prediction, some or all of the sensor-based measurements can be kept or discarded.

Particular implementations of the subject matter described in this disclosure can be implemented to realize one or more of the following potential advantages. Physiological characteristics (e.g., arterial compliance) can be derived to accurately estimate a difficult physiological parameter to obtain such as blood pressure in a simplified way. A unique compliance curve can be measured for each particular user at discrete points in time, and can be done passively while the user is asleep, rather than one continuous session requiring manual intervention or operation by the user. The data used to create the compliance curve may also be compatible with machine learning or deep learning implementations, where the data can be used as input to a machine learning or artificial intelligence model and further improve the accuracy of the blood pressure.

Additional details will follow after an initial description of relevant systems and technologies.

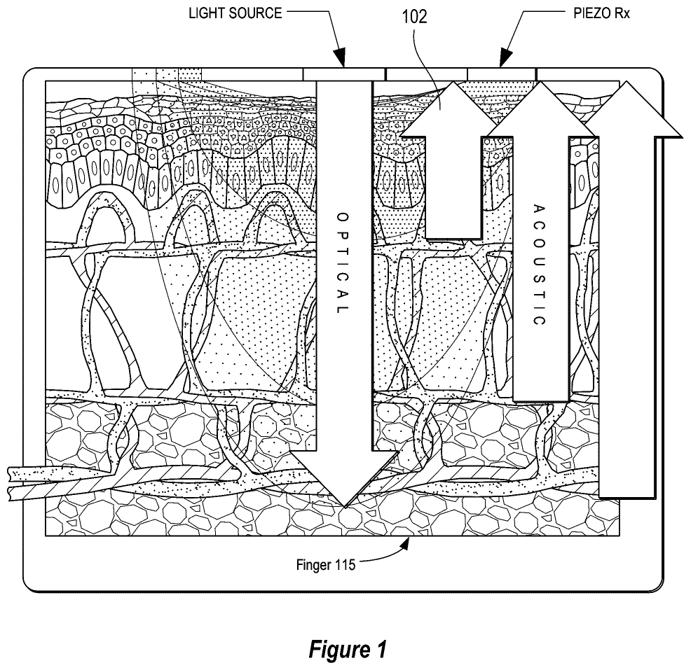

FIG. 1 shows an example of a blood pressure monitoring device based on photoacoustic plethysmography, which is referred to herein as PAPG. FIG. 1 shows the same examples of arteries, veins, arterioles, venules and capillaries inside a body part, which is a finger 115 in this example. In some examples, the light source shown in FIG. 1 may be coupled to a light source system (not shown) that is disposed remotely from the body part (e.g., finger 115). In some implementations, the light source may be an opening of an optical fiber or other waveguide. Such an opening may also be connected to an opening of an interface that is contactable with the body part. In some embodiments, the light source system may include one or more LEDs, one or more laser diodes, etc. In this example, the light source has transmitted light (in some examples, green, red, infrared, and/or near-infrared (NIR) light) that has penetrated the tissues of the finger 115 in an illuminated zone.

In the example shown in FIG. 1, blood vessels (and components of the blood itself) are heated by the incident light from the light source and are emitting acoustic waves 102. In this example, the emitted acoustic waves 102 include ultrasonic waves. According to this implementation, the acoustic wave emissions 102 are being detected by an ultrasonic receiver, which is a piezoelectric receiver in this example. Photoacoustic emissions 102 from the illuminated tissues, detected by the piezoelectric receiver, may be used to detect volumetric changes in the blood of the illuminated zone of the finger 115 that correspond to physiological data within the illuminated tissues of finger 115, such as heart rate waveforms. Although some of the tissue areas shown to be illuminated are offset from those shown to be producing photoacoustic emissions 102, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination.

One important difference between an optical technique such as a photoplethysmography (PPG)-based system the PAPG-based method of FIG. 1 is that the acoustic waves shown in FIG. 1 travel much more slowly than the reflected light waves involved in PPG. Accordingly, depth discrimination based on the arrival times of the acoustic waves shown in FIG. 1 is possible, whereas depth discrimination based on the arrival times of the light waves in PPG may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

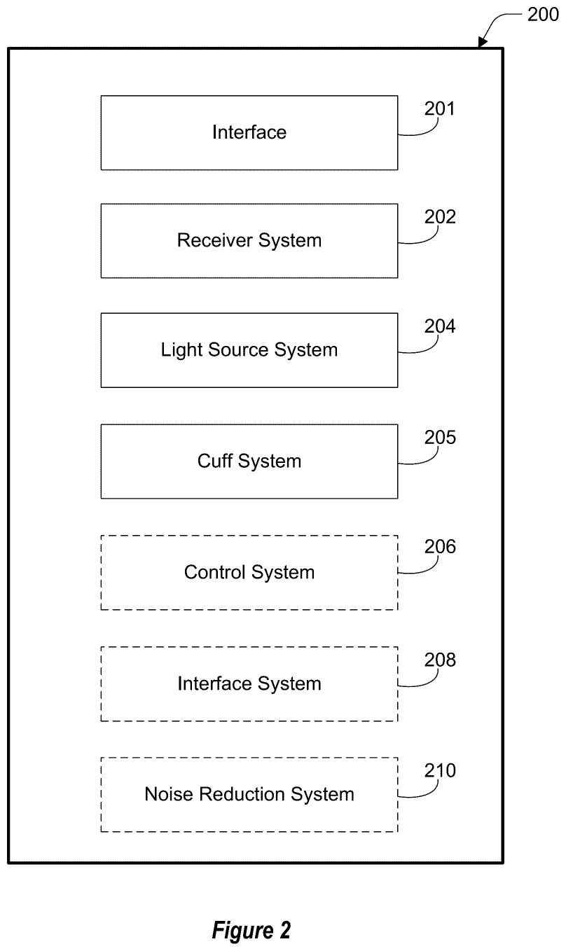

FIG. 2 is a block diagram that shows example components of a sensor apparatus 200 according to some implementations. In this example, the sensor apparatus 200 includes an interface 201, a receiver system 202, a light source system 204, and a cuff system 205. In some cases, a waveguide system may be included as a separate part of the sensor apparatus 200, or in some cases, may be part of the light source system 204. Some implementations of the sensor apparatus 200 may include a control system 206, an interface system 208, a noise reduction system 210, or a combination thereof.

Various examples of the interface 201 and various configurations of the receiver system 202 and the light source system 204 are disclosed herein. Some examples are described in more detail below.

In some embodiments, the interface 201, the receiver system 202, and the light source system 204 may be components of a photoacoustic (PAPG) sensor of the sensor apparatus 200. That is to say, in some embodiments, the sensor apparatus 200 may include a PAPG sensor and a cuff system 205. In various implementations described herein, the PAPG sensor and/or its components may operate in concert with the cuff system 205, e.g., timing the acquisition of photoacoustic measurements in relation to the operation of the cuff system 205, which will be described in greater detail following the example configurations of the example components of a sensor apparatus 200.

Some disclosed PAPG sensors described herein may include a platen, a light source system, and an ultrasonic receiver system. According to some implementations, the light source system may include a light source configured to produce and direct light. In some implementations, the platen may include an anti-reflective layer, a mirror layer, or combinations thereof. According to some implementations, the platen may have an outer surface, or a layer on the outer surface, with an acoustic impedance that is configured to approximate the acoustic impedance of human skin. In some implementations, the platen may have a surface proximate the ultrasonic receiver system, or a layer on the surface proximate the ultrasonic receiver system, with an acoustic impedance that is configured to approximate the acoustic impedance of the ultrasonic receiver system.

Some disclosed PAPG sensors described herein may include an interface, a light source system and an ultrasonic receiver system. Some such devices may not include a rigid platen. According to some implementations, the interface may be a physical, flexible interface constructed of one or more of suitable materials having a desired property or properties (e.g., an acoustic property such as acoustic impedance, softness of the material). In some implementations, the interface may be a flexible interface that can contact a target object that may be proximate to or contact the interface. There may be salient differences between such an interface and a platen. In some implementations, the light source system may be configured to direct light using one or more optical waveguides (e.g., optical fibers) configured to direct light toward a target object. According to some implementations, the interface may have an outer surface, or a layer on the outer surface, with an acoustic impedance that is configured to approximate the acoustic impedance of human skin. Such outer surface may have a contact portion that is contactable by a user or a body part of the user (e.g., finger, wrist). In some examples, the optical waveguide(s) may be embedded in one or more acoustic matching layers that are configured to bring the light transmitted by the optical waveguide(s) very close to tissue. The outer surface and/or other parts of the interface may be compliant, pliable, flexible, or otherwise at least partially conforming to the shape and contours of the body part of the user. In some implementations, the interface may have a surface proximate the ultrasonic receiver system, or a layer on the surface proximate the ultrasonic receiver system, with an acoustic impedance that is configured to approximate the acoustic impedance of the ultrasonic receiver system.

In some implementations in which the receiver system 202 includes an ultrasonic receiver system, the interface 201 may be an interface having a contact portion configured to make contact with a body part of a user such as the finger 115 shown in FIG. 1.

In some embodiments, the light source system 204 may, include one or more one or more light sources. In some implementations, the light source system 204 may include one or more light-emitting diodes. In some implementations, the light source system 204 may include one or more laser diodes. According to some implementations, the light source system 204 may include one or more vertical-cavity surface-emitting lasers (VCSELs). In some implementations, the light source system 204 may include one or more edge-emitting lasers. In some implementations, the light source system 204 may include one or more neodymium-doped yttrium aluminum garnet (Nd:YAG) lasers.

Hence, the light source system 204 may include, for example, a laser diode, a light-emitting diode (LED), or an array of either or both. The light source system 204 may be configured to generate and emit optical signals. The light source system 204 may, in some examples, be configured to transmit light in one or more wavelength ranges. In some examples, the light source system 204 may be configured to transmit light in a wavelength range of 500 to 600 nanometers (nm). According to some examples, the light source system 204 may be configured to transmit light in a wavelength range of 800 to 950 nm. According to some examples, the light source system 204 may be configured to transmit light in infrared or near infrared (NIR) region of the electromagnetic spectrum (about 700 to 2500 nm). In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm, or about 700 to 900 nm, or about 600 to 1100 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist. In some implementations, the light source system 204 may be configured to switch wavelengths to capture acoustic information from different depths, e.g., based on signal(s) from the control system 206.

In some implementations, the light source system 204 may be configured for emitting various wavelengths of light, which may be selectable to trigger acoustic wave emissions primarily from a particular type of material. For example, because the hemoglobin in blood absorbs near-infrared light very strongly, in some implementations the light source system 204 may be configured for emitting one or more wavelengths of light in the near-infrared range, in order to trigger acoustic wave emissions from hemoglobin. However, in some examples, the control system 206 may control the wavelength(s) of light emitted by the light source system 204 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system 202. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying range gate delays (RGDs)) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 204 may be configured for emitting a light pulse with a pulse width less than about 100 nanoseconds. In some implementations, the light pulse may have a pulse width between about 10 nanoseconds and about 500 nanoseconds or more. According to some examples, the light source system 204 may be configured for emitting a plurality of light pulses at a pulse repetition frequency between 10 Hz and 100 kHz. Alternatively, or additionally, in some implementations the light source system 204 may be configured for emitting a plurality of light pulses at a pulse repetition frequency between about 1 MHZ and about 100 MHZ. Alternatively, or additionally, in some implementations the light source system 204 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system 204 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 204. In some implementations, the light source system 204 may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

According to some examples, the light source system 204 may also include one or more light-directing elements configured to direct light from the light source system 204 towards the target object along the first axis. In some examples, the one or more light-directing elements may include at least one diffraction grating. Alternatively, or additionally, the one or more light-directing elements may include at least one lens.

In various configurations, the light source system 204 may incorporate anti-reflection (AR) coating, a mirror, a light-blocking layer, a shield to minimize crosstalk, etc.

The light source system 204 may include various types of drive circuitry, depending on the particular implementation. In some disclosed implementations, the light source system 204 may include at least one multi-junction laser diode, which may produce less noise than single-junction laser diodes. In some examples, the light source system 204 may include a drive circuit (also referred to herein as drive circuitry) configured to cause the light source system 204 to emit pulses of light at pulse widths in a range from 3 nanoseconds to 1000 nanoseconds. According to some examples, the light source system 204 may include a drive circuit configured to cause the light source system 204 to emit pulses of light at pulse repetition frequencies in a range from 1 kilohertz to 100 kilohertz.

In some example implementations, some or all of the one or more light sources of the light source system 204 may be disposed at or along an axis that is parallel to or angled relative to a central axis associated with the platen or interface 201. Optical signals may be emitted toward a target object (e.g., blood vessel), which may cause generation of ultrasonic waves by the target object. These ultrasonic waves may be detectable by one or more receiver elements of a receiver system 202.

Various examples of a receiver system 202 are disclosed herein, some of which may include ultrasonic receiver systems, optical receiver systems, or combinations thereof. In some implementations, the receiver system 202 includes an ultrasonic receiver system having the one or more receiver elements. In implementations that include an ultrasonic receiver system, the ultrasonic receiver and an ultrasonic transmitter may be combined in an ultrasonic transceiver. In some examples, the receiver system 202 may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 202 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 202 may be, or may include, an ultrasonic receiver array. In some examples, the sensor apparatus 200 may include one or more separate ultrasonic transmitter elements or one or more separate arrays of ultrasonic transmitter elements. In some examples, the ultrasonic transmitter(s) may include an ultrasonic plane-wave generator.

In some implementations, at least portions of the sensor apparatus 200 (for example, the receiver system 202, the light source system 204, or both) may include one or more sound-absorbing layers, acoustic isolation material, light-absorbing material, light-reflecting material, or combinations thereof. In some examples, acoustic isolation material may reside between the light source system 204 and at least a portion of the receiver system 202. In some examples, at least portions of the sensor apparatus 200 (for example, the receiver system 202, the light source system 204, or both) may include one or more electromagnetically shielded transmission wires. In some such examples, the one or more electromagnetically shielded transmission wires may be configured to reduce electromagnetic interference from the light source system 204 that is received by the receiver system 202.

In some embodiments, the sensor apparatus 200 may include a cuff system 205. The cuff system 205 may include, in some implementations, a pump, a bladder, and/or a pressure sensor. Further components of the cuff system 205 may include a vent, a pump driver, a controller, a printed circuit board, a temperature sensor, a memory, a processor, a valve, a nozzle, a tube, a power source or battery, a physical structure (e.g., a wearable structure, a housing, a cuff), or some combination thereof.

The pump may be configured to flow air into the bladder to cause positive pressure within the bladder. The bladder may be an air bag constructed to be pressurized by the air contained therein. In some configurations, a pump driver (including, e.g., circuitry, logic, processor) may control the pump and cause the influx of air into the bladder. Voltage may be applied to the pump by the pump driver or the controller to control the influx of air. In some implementations, the pressure caused by the air may be constant over a period of time. Such pressure may be changed incrementally by adjusting a voltage level. Hence, external pressure may be applied and held at discrete pressure levels. A vent may also be controlled by the controller to allow air to escape the bladder, which reduces the pressure. The pressure sensor may be used to detect the pressure inside the bladder, resulting in pressure data for operations described herein. The pressure can be adjusted to a prescribed level based on the detected pressure.

The control system 206 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 206 also may include (and/or be configured for communication with) one or more memory devices, such as one or more random access memory (RAM) devices, read-only memory (ROM) devices, etc. Accordingly, the sensor apparatus 200 may have a memory system that includes one or more memory devices, though the memory system is not shown in FIG. 2. The control system 206 may be configured for receiving and processing data from the receiver system 202, e.g., as described below. If the sensor apparatus 200 includes an ultrasonic transmitter, the control system 206 may be configured for controlling the ultrasonic transmitter. In some implementations, functionality of the control system 206 may be partitioned between one or more controllers or processors, such as a dedicated sensor controller and an applications processor of a mobile device.

In some examples, the control system 206 may be communicatively coupled to the light source system 204 and configured to control the light source system to emit light towards a target object on an outer surface of the interface 201. In some such examples, the control system 206 may be configured to receive signals from the ultrasonic receiver system (including one or more receiver elements) corresponding to the ultrasonic waves generated by the target object responsive to the light from the light source system. In some examples, the control system 206 may be configured to identify one or more blood vessel signals, such as arterial signals or vein signals, from the ultrasonic receiver system. In some such examples, the one or more arterial signals or vein signals may be, or may include, one or more blood vessel wall signals corresponding to ultrasonic waves generated by one or more arterial walls or vein walls of the target object. In some such examples, the one or more arterial signals or vein signals may be, or may include, one or more arterial blood signals corresponding to ultrasonic waves generated by blood within an artery of the target object or one or more vein blood signals corresponding to ultrasonic waves generated by blood within a vein of the target object. In some examples, the control system 206 may be configured to determine or estimate one or more physiological parameters or cardiac features based, at least in part, on one or more arterial signals, on one or more vein signals, or on combinations thereof. According to some examples, a physiological parameter may be, or may include, blood pressure. In some approaches, blood pressure can be estimated based at least on PWV, as will be discussed below.

In further examples, the control system 206 may be communicatively coupled to the receiver system 202. The receiver system 202 may be configured to detect acoustic signals from the target object. The control system 206 may be configured to select at least one of a plurality of receiver elements of the receiver system 202. Such selected receiver element(s) may correspond to the best signals from multiple receiver elements. In some embodiments, the selection of the at least one receiver element may be based on information regarding detected acoustic signals (e.g., arterial signals or vein signals) from the plurality of receivers. For example, signal quality or signal strength (based, e.g., on signal-to-noise ratio (SNR)) of some signals may be relatively higher than some others or above a prescribed threshold or percentile, which may indicate the best signals. In some implementations, the control system 206 may also be configured to, based on the information regarding detected acoustic signals, determine or estimate at least one characteristic of the blood vessels such as PWV (indicative of arterial stiffness), arterial dimensions, or both.

Some implementations of the sensor apparatus 200 may include an interface system 208. In some examples, the interface system 208 may include a wireless interface system. In some implementations, the interface system 208 may include a user interface system, one or more network interfaces, one or more interfaces between the control system 206 and a memory system and/or one or more interfaces between the control system 206 and one or more external device interfaces (e.g., ports or applications processors), or combinations thereof. According to some examples in which the interface system 208 is present and includes a user interface system, the user interface system may include a microphone system, a loudspeaker system, a haptic feedback system, a voice command system, one or more displays, or combinations thereof. According to some examples, the interface system 208 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 208 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some implementations, the force sensor system may be mechanically coupled to a platen. In some such examples, the force sensor system may be integrated into circuitry of the ultrasonic fingerprint sensor. In some examples, the interface system 208 may include an optical sensor system, one or more cameras, or a combination thereof.

According to some examples, the sensor apparatus 200 may include a noise reduction system 210. For example, the noise reduction system 210 may include one or more mirrors that are configured to reflect light from the light source system 204 away from the receiver system 202. In some implementations, the noise reduction system 210 may include one or more sound-absorbing layers, acoustic isolation material, light-absorbing material, light-reflecting material, or combinations thereof. In some examples, the noise reduction system 210 may include acoustic isolation material, which may reside between the light source system 204 and at least a portion of the receiver system 202, on at least a portion of the receiver system 202, or combinations thereof. In some examples, the noise reduction system 210 may include one or more electromagnetically shielded transmission wires. In some such examples, the one or more electromagnetically shielded transmission wires may be configured to reduce electromagnetic interference from circuitry of the light source system, receiver system circuitry, or combinations thereof, that is received by the receiver system.

In some embodiments, the sensor apparatus 200 may be a wearable device configured to be worn by a user, e.g., around the wrist, finger, arm, leg, ankle, or another appendage, or another portion of the body. In an example implementation, the sensor apparatus 200 may have the form of a wristwatch and can be worn around the wrist. The cuff system 205 may apply pressure around the wrist, while the skin at the wrist makes contact via the interface 201 and photoacoustic measurements can be taken by virtue of operating the receiver system 202 and the light source system 204. However, the embodiments described herein are not so limited. In certain cases, the components of the sensor apparatus 200 may not all be worn. For instance, the cuff system 205 may be worn around an appendage, similar to a sphygmomanometer, but other components such as the receiver system 202 and the light source system 204 may be in a separate PAPG sensor component and/or not be in a wearable chassis in order to collect photoacoustic measurements.

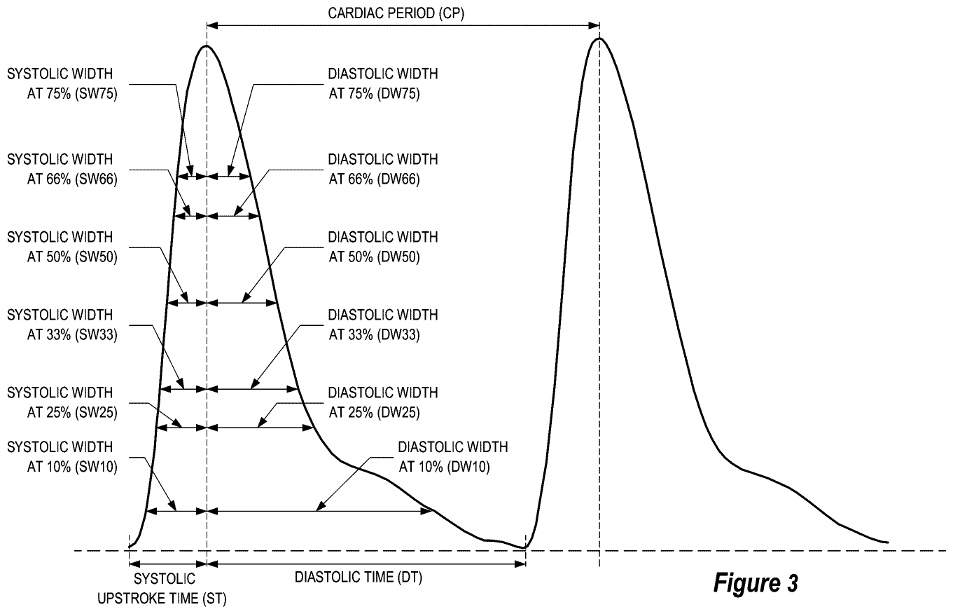

FIG. 3 shows examples of heart rate waveform (HRW) features that may be extracted according to some implementations. The horizontal axis of FIG. 3 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 3 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75. (DW66+SW66), DW66/SW66, (DW50+SW50), DW50/SW50, (DW33+SW33), DW33/SW33, (DW25+SW25), DW25/SW25 and/or (DW10+SW10). DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

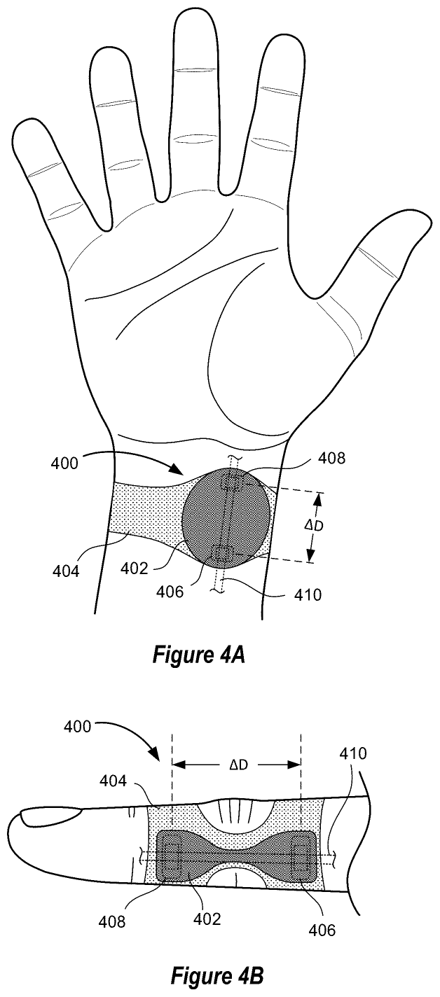

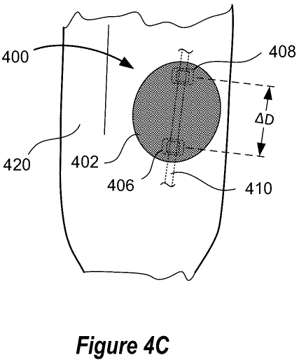

In some implementations, the monitoring device can be positioned around a wrist of a user with a strap or band, similar to a watch or fitness/activity tracker. FIG. 4A shows an example device 400 designed to be worn around a wrist according to some implementations. In some embodiments, the example device 400 may include the sensor apparatus 200 so as to allow components of the sensor apparatus 200 to interact with the user, e.g., via the skin of the user. In the illustrated example, the monitoring device 400 includes a housing 402 integrally formed with, coupled with or otherwise integrated with a wristband 404. The first and the second arterial sensors 406 and 408 may, in some instances, each include an instance of the ultrasonic receiver system and a portion of the light source system that are described above. In this example, the example device 400 is coupled around the wrist such that the first and the second arterial sensors 406 and 408 within the housing 402 are each positioned along a segment of the radial artery 410 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial sensors 406 and 408 are separated by a fixed distance ΔD. In some other implementations, the example device 400 can similarly be designed or adapted for positioning around a forearm, an upper arm, an ankle, a lower leg, an upper leg, or a finger (all of which are hereinafter referred to as “limbs”) using a strap or band.

FIG. 4B shows an example device 400 designed to be worn on a finger according to some implementations. The first and the second arterial sensors 406 and 408 may, in some instances, each include an instance of the ultrasonic receiver and a portion of the light source system that are described above.

In some other implementations, the devices disclosed herein can be positioned on a region of interest of the user without the use of a strap or band. For example, the first and the second arterial sensors 406 and 408 and other components of the monitoring device can be enclosed in a housing that is secured to the skin of a region of interest of the user using an adhesive or other suitable attachment mechanism (an example of a “patch” monitoring device).

FIG. 4C shows an example device 400 designed to reside on an earbud according to some implementations. According to this example, the monitoring device 400 is coupled to the housing of an earbud 420. The first and second arterial sensors 406 and 408 may, in some instances, each include an instance of the ultrasonic receiver and a portion of the light source system that are described above.

### Example Signal Acquisition and Analyses

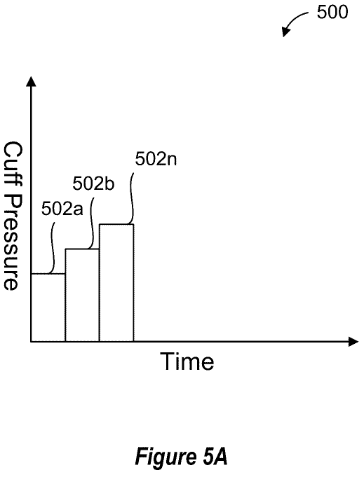

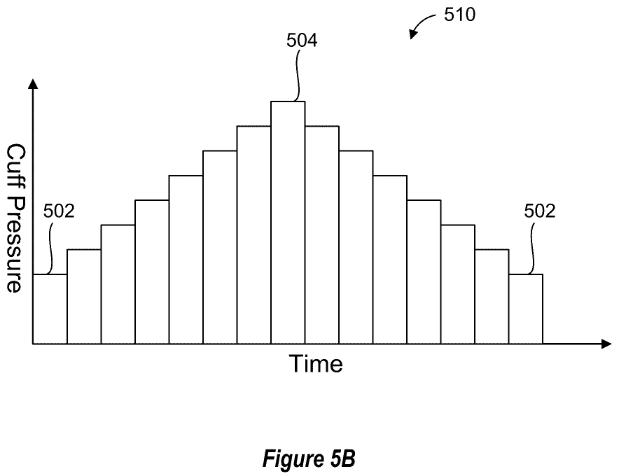

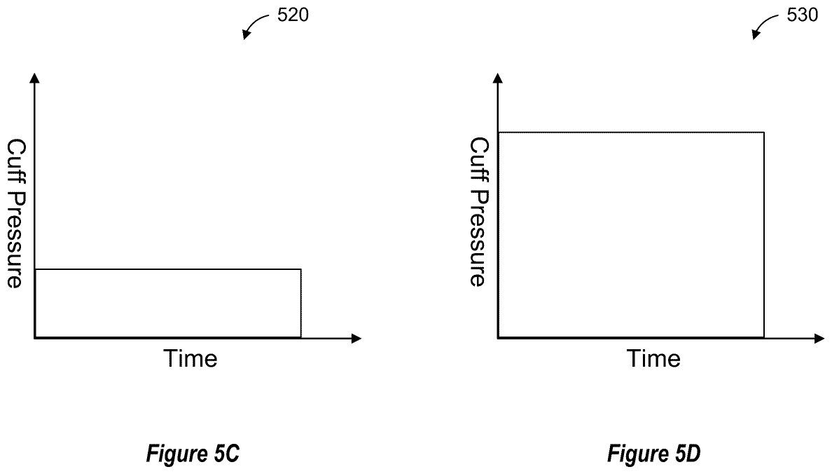

As noted elsewhere, a sensor apparatus (e.g., sensor apparatus 200) may be worn at least partially by a user in order to apply pressure at different discrete pressure levels and to obtain photoacoustic signals and measurements. Such pressure applied by the sensor apparatus may be referred to herein as external pressure or externally applied pressure. In some implementations, segments of data may be collected at multiple external pressures, e.g., as applied by a cuff system. For example, photoacoustic signals may be acquired by the sensor apparatus (e.g., using the receiver system 202, and the light source system 204) over periods of time corresponding to the external pressures. That is, photoacoustic measurements are obtained for each pressure level to acquire information about a target object (e.g., blood vessel). FIGS. 5A-5D are illustrative of this and now discussed.

FIG. 5A is a diagram of a graph 500 showing an example of externally applied pressures (e.g., with a cuff system) over time according to some implementations. In this example, three discrete pressure levels are shown: a first pressure level 502a, a second pressure level 502b, and a third pressure level 502n. The cuff system may apply these pressures at a constant level over a set period of time. In a non-limiting example, the first pressure level 502a may be 30 mmHg or approximately 30 mmHg applied during a first time period, the second pressure level 502b may be 40 mmHg or approximately 40 mmHg applied during a second time period, and the third pressure level 502n may be 50 mmHg or approximately 50 mmHg applied during a third time period. The pressure levels may be pre-set, or selected based on the tensive state of a user (e.g., hypotensive, hypertensive, normotensive). That is, users with different blood pressures may have different target cuff inflation pressures or ranges. Approximate pressure levels may refer to pressure levels having an associated range or error of, for example, ±1 mmHg, ±2 mmHg, ±3 mmHg, etc. or ±0.1%, ±0.2%, ±0.5%, ±1%, etc. Each time period may last from a few seconds or longer (e.g., 3 seconds, 10 seconds, 60 seconds, or more). The pressures may be low enough (e.g., 100 mmHg or below, 90 mmHg or below, or other low pressures) such that the user does not feel discomfort but high enough to cause sufficient pressure on the target object, e.g., a blood vessel.

In each of these time periods, photoacoustic signals may be obtained and stored. Together, the photoacoustic signals from multiple pressure levels can be analyzed to obtain highly correlated features and extract salient information, such as other compliance information. Other types of information (e.g., arterial dimensions, spatial parameters such as area or volume, distensibility/PWV, and others) may also be determined from the correlated features.

In some cases, there may be a transition period between each pressure level, which is not shown for simplicity. During a transition period, the pressure level may change, e.g., from 30 to 40 mmHg or 40 to 50 mmHg (or decreasing from 50 to 40 or 30 mmHg), each of which may take a few seconds or more. Longer transition periods may make the change in applied pressure less noticeable to the user, contributing to the comfort of wearing the device.

FIG. 5B is a diagram of a graph 510 showing another example of externally applied pressures over time according to some implementations. In this example, the pressure level may start at a first pressure level 502, incrementally increase over time (similar to how such increase was performed in graph 500). The pressure level may reach a maximum pressure level 504, and incrementally decrease over time. Each incremental increase or decrease may be associated with a transition period where the bladder is inflated or deflated, respectively. In a non-limiting example, the pressure may progress from an initial first pressure level 502 of 30 mmHg or approximately 30 mmHg during a first time period, up to a maximum pressure level 504 of 100 mmHg or approximately 100 mmHg during a subsequent time period, and back to the first pressure level 502 of 30 mmHg or approximately 30 mmHg during a subsequent time period. However, any sequence of pressure levels may be applied. In one of myriad approaches, the maximum pressure level 504 may be 90 mmHg (or less); the range of pressures applied may thus be 30 to 90 mmHg (or approximately so). In another approach, the first pressure level 502 may be 40 mmHg (or more). In each of these time periods, photoacoustic signals may be obtained and stored.

The sequence shown in graph 510 may be useful for calibration for the individual user. More specifically, by going from a wider range of pressure levels, in this case 30 to 100 mmHg, a larger set of photoacoustic measurements can be acquired as opposed to a smaller range or a constant pressure level such as in FIG. 5C as discussed below.

FIGS. 5C and 5D are diagrams of graphs 520, 530 showing examples of a substantially constant externally applied pressure (e.g., with a cuff system) over time according to some implementations are illustrated. Graph 520 shows a relatively low constant pressure (e.g., about 40 mmHg or less), whereas graph 530 shows a relatively high constant pressure (e.g., 90 mmHg or thereabout). In some scenarios, substantially constant may refer to an associated range or error of, for example, ±1 mmHg, ±2 mmHg, ±3 mmHg, etc. or ±0.1%, ±0.2%, ±0.5%, ±1%, etc. The substantially constant pressure may be applied for a period of time, such as 3 seconds, 5 seconds, 10 seconds, 30 seconds, 60 seconds, or more. Once a so-called compliance curve is determined (e.g., possibly through calibration with pressure levels shown in graph 510 of FIG. 5B), applying a constant pressure may allow determination of the physiological parameter of the user such as blood pressure.

Depending on the pressure applied to the blood vessel, its characteristics may change. Characteristics of the blood vessel may include, for example, arterial compliance, distension, stiffness, dimensions (e.g., diameter), and PWV. For instance, change may occur because the pressure causes the blood vessel to be flatter during at least a portion of a pulse. The blood vessel may have an elliptical or approximately elliptical shape as a result, and have a major axis and a minor axis.

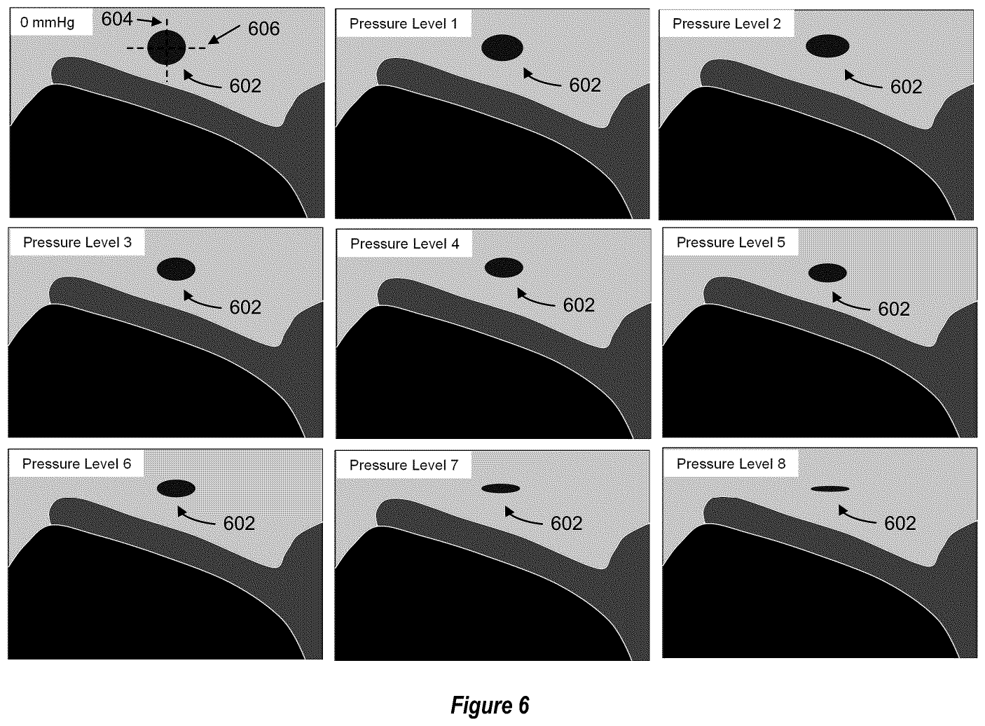

FIG. 6 depicts ultrasound images of a blood vessel 602 at different levels of pressure to illustrate the elliptical shape and dimensions based on externally applied pressure. As can be seen, the blood vessel 602 is substantially circular. In some cases, the blood vessel 602 may still have elliptical features, where a diameter across one axis (major axis 606) is slightly longer than a diameter across the other axis (minor axis 604). Increasing pressure levels from 1 through 8 (above 0 mmHg) are shown. For example, Pressure Level 1 may correspond to 20 mmHg. As greater levels of external pressure are applied, the difference between the diameter across the minor axis 604 and the diameter across the major axis 606 becomes greater. That is, the blood vessel 602 appears flatter at greater external pressure levels, and the blood vessel 602 becomes more elliptical. Thus, FIG. 6 shows that the dimensions of the blood vessel 602 varies with external pressure.

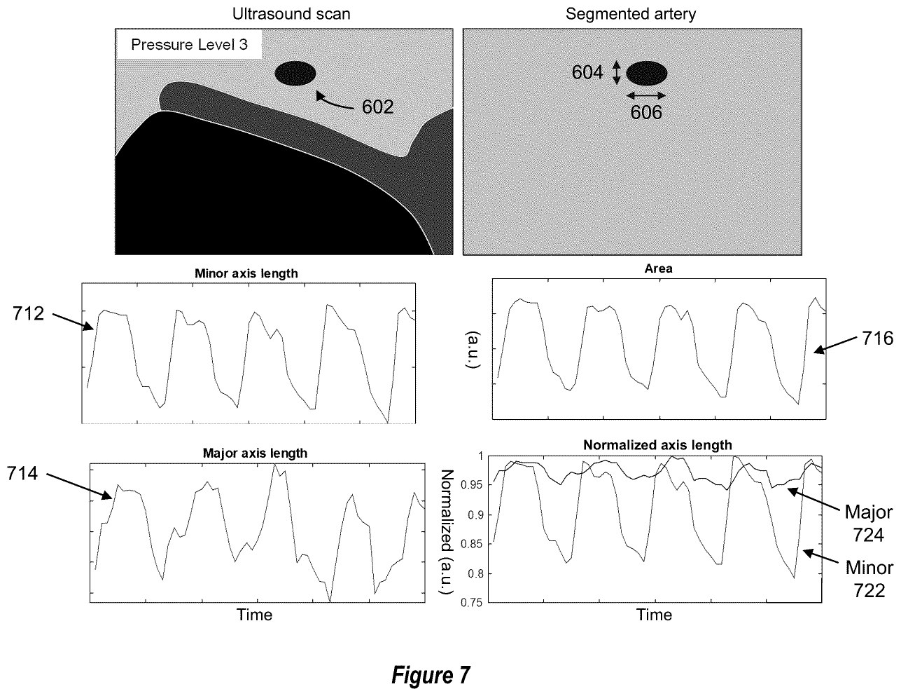

FIG. 7 depicts dimensions and spatial measurements of a blood vessel 602 experiencing a constant external pressure over time. The pressure level applied may correspond to an amount of pressure (e.g., 60 mmHg) that causes the blood vessel 602 to form a clearly elliptical shape. Over time, as blood is moved through the cardiovascular system of the user, the blood vessel 602 may still distend due to force of the blood pulsing through. The minor axis length 712 may change over time along with the major axis length 714. It can be seen from the differences in normalized minor axis length 722 and normalized major axis length 724 that the distension of the blood vessel 602 along the minor axis is more pronounced than along the major axis. Consequently, the cross-sectional area 716 of the blood vessel 602 may change at corresponding times (e.g., during pulses).

At least some of the aforementioned characteristics of the blood vessel can be captured using photoacoustic sensing, where photoacoustic signals may be obtained by the sensor apparatus (e.g., using the receiver system 202, and the light source system 204) and stored. Examples of characteristics that can be derived from photoacoustic sensing include arterial dimensions and distension. Other characteristics and parameters (e.g., compliance, PWV) may be derived from the photoacoustic measurements.

In some approaches, “beamformed” images can be generated from photoacoustic signals. Multiple channels or sources of photoacoustic data can be used to generate such an image. A beamformed image may refer herein to an image generated as a result of a “delay-and-sum” process with multiple receiver elements of a receiver system. More specifically, a delay could be applied to an ultrasonic receiver signal by performing a correlation operation on input ultrasonic receiver signals. For example, a control system may perform a correlation operation on first and second ultrasonic different receiver signals, and may determine that by applying a first time shift to the first ultrasonic receiver signal, the first ultrasonic receiver signal would be strongly correlated with a third ultrasonic receiver signal. Similarly, the control system may perform a correlation operation on second and third ultrasonic receiver signals, and may determine that by applying a second time shift to the second ultrasonic receiver signal, the second ultrasonic receiver signal would be strongly correlated with the third ultrasonic receiver signal.

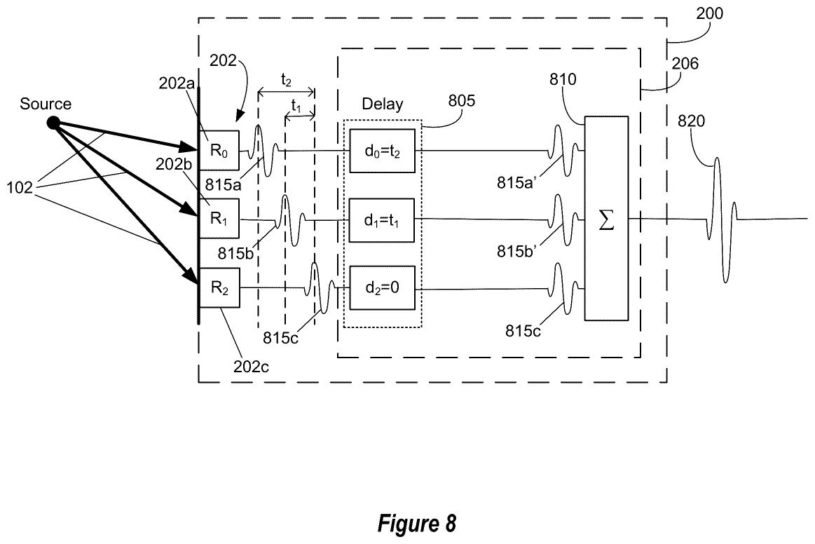

FIG. 8 shows an example of an apparatus that is configured to perform a receiver-side beamforming process. In this example, the receiver-side beamforming process is a delay-and-sum beamforming process. As with other disclosed examples, the types, numbers, sizes and arrangements of elements shown in FIG. 8 and described herein, as well as the associated described methods, are merely examples.

In this example, a source is shown emitting ultrasonic waves 102, which are detected by active ultrasonic receiver elements 202a, 202b and 202c of an array of ultrasonic receiver elements. The array of ultrasonic receiver elements is part of an ultrasonic receiver system 202. The ultrasonic waves 102 may, in some examples, correspond to the photoacoustic response of a target object to light emitted by a light source system 204 of the sensor apparatus 200. In this example, the active ultrasonic receiver elements 202a, 202b and 202c provide ultrasonic receiver signals 815a, 815b and 815c, respectively, to the control system 206.

According to this example, the control system 206 includes a delay module 805 and a summation module 810. In this example, the delay module 805 is configured to determine whether a delay should be applied to each of the ultrasonic receiver signals 815a. 815b and 815c, and if so, what delay will be applied. According to this example, the delay module 805 determines that a delay d₀ of t₂ should be applied to the ultrasonic receiver signal 815a, that a delay d₁ of t₁ should be applied to the ultrasonic receiver signal 815b and that no delay should be applied to the ultrasonic receiver signal 815c. Accordingly, the delay module 805 applies a delay of t₂ to the ultrasonic receiver signal 815a, producing the ultrasonic receiver signal 815a′, and applies a delay of t₁ to the ultrasonic receiver signal 815b, producing the ultrasonic receiver signal 815b′.

In some examples, the delay module 805 may determine what delay, if any, to apply to an ultrasonic receiver signal by performing a correlation operation on input ultrasonic receiver signals. For example, the delay module 805 may perform a correlation operation on the ultrasonic receiver signals 815a and 815c, and may determine that by applying a time shift of t₂ to the ultrasonic receiver signal 815a, the ultrasonic receiver signal 815a would be strongly correlated with the ultrasonic receiver signal 815c. Similarly, the delay module 805 may perform a correlation operation on the ultrasonic receiver signals 815b and 815c, and may determine that by applying a time shift of t₁ to the ultrasonic receiver signal 815b, the ultrasonic receiver signal 815b would be strongly correlated with the ultrasonic receiver signal 815c.

According to this example, the summation module 810 is configured to sum the ultrasonic receiver signals 815a′, 815b′ and 815c, producing the summed signal 820. One may observe that the amplitude of the summed signal 820 is greater than the amplitude of any one of the ultrasonic receiver signals 815a, 815b or 815c. In some instances, the signal-to-noise ratio (SNR) of the summed signal 820 may be greater than the SNR of any of the ultrasonic receiver signals 815a, 815b or 815c.

Put another way, according to this example, the control system may be configured to sum the first time-shifted ultrasonic receiver signal, the second time-shifted ultrasonic receiver signal, and the third ultrasonic receiver signal, producing a summed signal. The amplitude of the summed signal may be greater than the amplitude of any one of the first, second, or third ultrasonic receiver signal. The signal-to-noise ratio (SNR) of the summed signal may be greater than the SNR of any of the first, second, or third ultrasonic receiver signal. Hence, cleaner, stronger, and less noisy signals may be obtained by using multiple receiver elements and time-shifting certain ultrasonic signals.

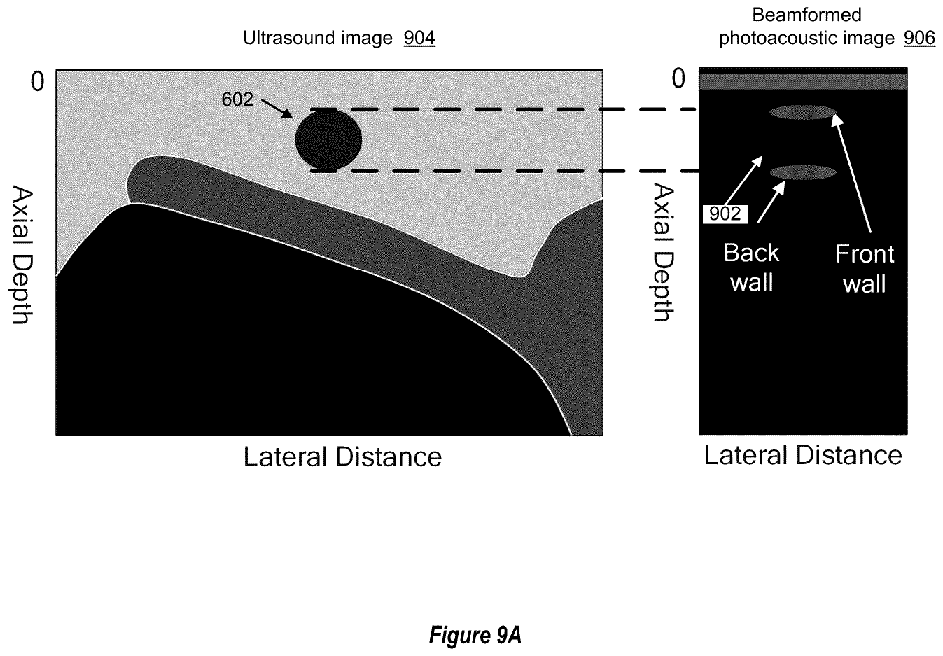

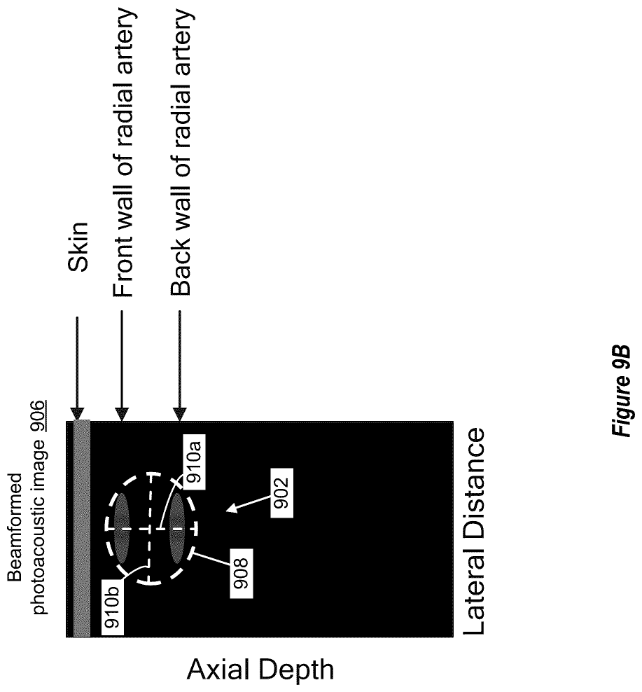

FIG. 9A depicts a beamformed photoacoustic image 906 based on an ultrasonic image 904 of a blood vessel 602, 902, according to some implementations. The ultrasonic image 904 may depict an image at an arbitrary low-contact pressure. Axial depth (how deep into the tissue) and lateral distance (how wide) of the blood vessel 902 can be determined from the ultrasonic image 904 to the photoacoustic image 906 by correlating edge portions of the blood vessel 602 in the ultrasonic image 904 to a back wall and a front wall of the blood vessel 902 shown in the beamformed photoacoustic image 906, as indicated by dotted lines.

FIG. 9B depicts the beamformed photoacoustic image 906 in more detail. The front wall and the back wall of the blood vessel 902 can be detected in the beamformed photoacoustic image 906 based on signal strength. In some implementations, an ellipsis or circular boundary 908 can be fitted onto the beamformed photoacoustic image 906, and a major axis 910a and a minor axis 910b may be determined. Half of the major axis 910a is referred to as a semi-major axis, and half of the minor axis 910b is referred to as a semi-minor axis, collectively semi-axes. In some example approaches, image processing, an algorithmic process, or machine learning and training can be used to determine the boundary for the blood vessel 902, from which axial depth and lateral distance can be used to estimate the major and minor axes. The major and minor axes of the blood vessel 902 are dimensions that can be used to determine cross-sectional area and/or volume of the blood vessel 902, whose salience will become apparent in the discussions occurring below.

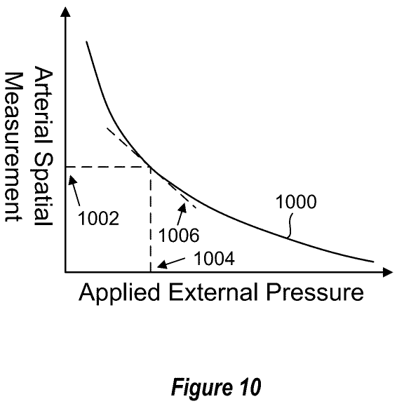

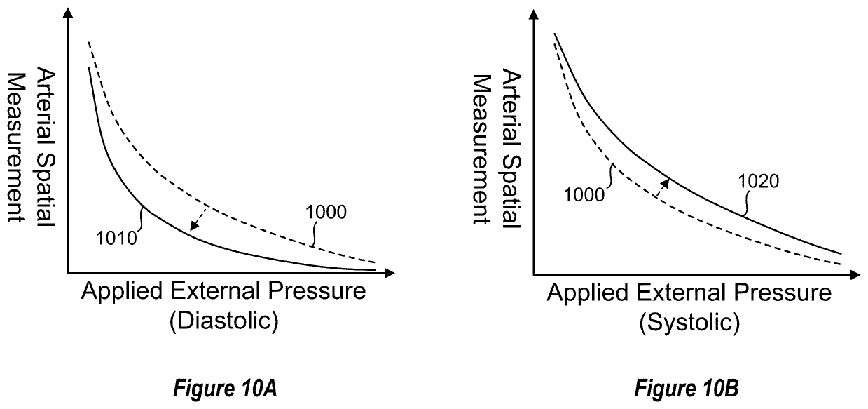

Now referring to FIG. 10, a graph is shown of an example curve 1000 of arterial spatial measurement as a function of applied external pressure, according to some implementations. This curve may herein be referred to as a compliance curve. In some approaches, by inflating a cuff of a sensor apparatus (such as sensor apparatus 200 embodied as a wearable device) and taking photoacoustic measurements from a target object (e.g., blood vessel) during different discrete pressure levels, dimensions such as diameter (e.g., major axis and/or minor axis) of the target object can be obtained. In some examples, arterial diameters at respective pressure levels may be obtained using photoacoustic measurements from a blood vessel.

In some implementations, spatial measurements of the blood vessel, such as area (cross-sectional) or volume, can be calculated or inferred from the arterial diameters. More specifically, for a circular or substantially circular blood vessel, cross-sectional area can be estimated using the well-known formula of A_{circle}=πr², and volume can be estimated using V_{circle}=πr²l, where r is the radius (half of obtained arterial diameter), and l is the length of a segment of interest of the blood vessel. The value of l may be an arbitrary constant, within a minimum and/or maximum length selected for accurate estimation of fluid dynamics of the blood vessel, based on a dimension of the wearable device, and/or based on an actual photoacoustic response of the segment of interest. In some approaches, length may not be taken into account, as it will be constant, e.g., where the parameter of interest is based on area change or ratio of initial to final volume.

For an elliptical blood vessel, where applied external pressure greater than a threshold amount (e.g., 20 mmHg or higher) causes a singular diameter to be considered inaccurate, elliptical area of the cross section and elliptical volume can be derived as follows: Aₑₗₗᵢₚₛₑ=π*a*b, where a is the radius (half of obtained arterial diameter) of the major axis and b is the radius of the minor axis. Put differently, a is the length of semi-minor axis, and b is the length of the semi-minor axis. In some approaches, Vₑₗₗᵢₚₛₑ=π*a*b*l, where I is the length of the segment of interest of the blood vessel. In alternative approaches, V_{ellipsoid}=(4/3)*π*a*b*c, where c is the length of the third semi-axis along the segment of interest of the blood vessel. The value of c or l may be an arbitrary constant, within a minimum and/or maximum length selected for accurate estimation of fluid dynamics of the blood vessel, based on a dimension of the wearable device, and/or based on an actual length of the segment of interest.

Since the area and volume of a circular or elliptical blood vessel are related by a constant, either of the derived spatial measurement, e.g., area or volume, could be used to derive the curve 1000 and result in similar curve profiles. The arterial spatial measurement can broadly refer herein to cross-sectional area or volume associated with the blood vessel. In some approaches, the curve 1000 may be fitted to the data points representing the derived spatial measurements corresponding to the applied external pressures. A curve such as an exponential curve, a polynomial curve (quadratic, cubic, fourth order, etc.), logarithmic curve, etc. (or a line) may be determined. A normalized curve (ranging from 0 to 1 on either or both axes) could also be derived. In some approaches, a trained machine learning model can be used to generate the curve 1000. For instance, a dataset of applied external pressures, and arterial dimensions (e.g., diameters, semi-axes) and/or arterial spatial measurements (e.g., cross-sectional area, volume) can be inputted to a machine learning model trained on “ground truth” curves, which could then generate the curve 1000 based on the inputs.

Reviewing the curve 1000, it can be seen that the greater the applied pressure, the flatter the blood vessel, and the smaller the area or volume. Hence, a compliance curve has a downward trend with greater external pressure. The relationship between the arterial volume or arterial area and the applied pressure at can be determined at each measured pressure level on the curve 1000. A corresponding spatial measurement 1002 could be identified at a given applied external pressure level 1004. An advantage of applying external pressure is that signal quality is improved, e.g., from having a substantially constant pressure applied by a cuff.

Moreover, a slope 1006 at the point of the curve 1000 that corresponds to the given applied external pressure level 1004 can be identified. This slope 1006 provides compliance information for the user. Compliance (C) can also be expressed as C=dV/dP or C=dA/dP, where P is the applied external pressure. Compliance information is based on elastic properties of a blood vessel and is determinant of the speed of pulse pressure waves. Compliance can be different for each person based on physiology, activity, chronic conditions, etc., and will change based on the state of the individual. For instance, the user may be relaxed, agitated, in a post-exercise state, or sleeping, all of which can affect blood flow, heart rate, blood pressure, etc. These states of the user will be associated with different arterial compliance values. Therefore, compliance information obtained for each user individually across various pressure levels provides a larger set of data, range of measurements, and features.

There is also a possibility of intermittent recalibration of the compliance curve and/or the sensor apparatus to account for any changes in the user's physiology or the placement of the sensor apparatus and thus the location of the blood vessel. Recalibration can also correct any drifts in sensor systems or cuff systems. Depending on the approach, recalibration may be performed at various time intervals. In some examples, recalibration may occur every two to three months, every month, every week, every day (e.g., immediately before or during sleep so as to minimize interruption of the user's activities while awake), or manually. Myriad variables exist for selecting a recalibration period.

In some variations, more than one compliance curve may be determined separately, e.g., for systolic diameters and diastolic diameters measured during systole and diastole, respectively. Systolic pressure is the maximum blood pressure during ventricular contraction (systole), and diastolic pressure is the minimum pressure recorded just prior to the next contraction (diastole). During stages of the heart's rhythm where blood pressure is relatively reduced, such as during diastole, dimensions of the blood vessel may be relatively smaller because of the reduced force experienced within the blood vessel. On the other hand, during heightened blood pressures such as during systole, dimensions of the blood vessel may be greater because of the greater force experienced within the blood vessel.

FIG. 10A illustrates an example of a curve 1010 of arterial spatial measurements corresponding to external pressures applied during diastole, compared with curve 1000, according to some implementations. FIG. 10B illustrates an example of a curve 1020 of arterial spatial measurements corresponding to external pressures applied during systole, compared with curve 1000, according to some implementations. The profile of each of the curves 1010, 1020 is similar to that of curve 1000, which in view of curves 1010, 1020 may represent average pressures or have wider error bars associated with spatial measurements. Such multiple compliance curves can provide context that can be evaluated to extract additional compliance information.

However, additional information is needed to determine PWV and blood pressure.

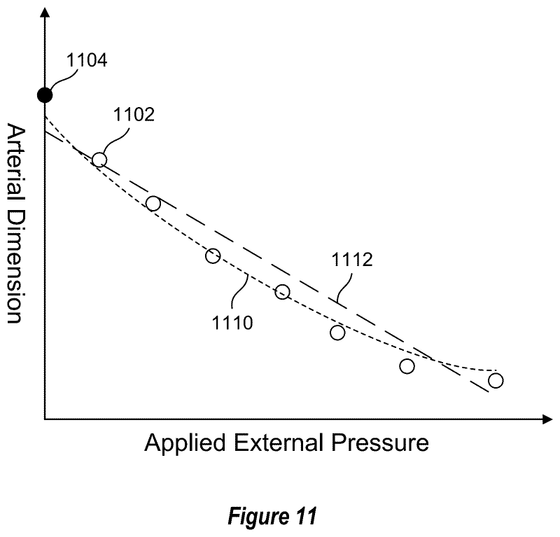

FIG. 11 is a graph of an example set of data points of arterial dimensions as a function of applied external pressure, according to some implementations. As mentioned previously, dimensions (e.g., diameter of a target object such as a blood vessel) can be measured, e.g., via photoacoustic signals collected at different external pressures. Each hollow circle represents an associated arterial dimension 1102 for a respective external counter pressure applied, e.g., via a cuff worn by the user (e.g., cuff system 205 of a sensor apparatus 200). However, it is difficult to obtain the arterial dimension at zero applied external pressure, as contact with the skin is required to obtain photoacoustic signals for determining the arterial dimension. Any air gap would not result in accurate photoacoustic measurements.

To this end, in some embodiments, photoacoustic measurements at several external pressures and the corresponding dimension measurements can be used to extrapolate the arterial dimension at zero pressure. In some approaches, a curve 1110 may be fitted to the arterial dimension measurements 1102, such as an exponential curve, a polynomial curve (quadratic, cubic, fourth order, etc.), logarithmic curve, etc. In some approaches, a line 1112 may be fitted to the arterial dimension measurements 1102. Based on the extrapolation, a zero-pressure arterial dimension 1104 may be determined.

In some embodiments, the arterial dimension at zero pressure may be determined using a machine learning model trained to output the arterial dimension based on a plurality of discrete pressures and/or a corresponding plurality of arterial dimensions as input.

This arterial dimension, e.g., the zero-pressure arterial diameter, can be the basis for a zero-pressure spatial measurement such as a zero-pressure cross-sectional area (or volume). This zero-pressure spatial measurement can provide additional information to estimate blood pressure as described below.

In some embodiments, the shape of the heart rate waveform (HRW) at different external pressure levels may also provide useful information or context. Since the shape of the HRW will change with different applied external pressures, HRW features corresponding to high, low, or no external applied pressure my aid in blood pressure estimation. For example, evaluating obtained HRW features against known HRW features at corresponding known arterial dimensions or applied pressures can validate that the arterial dimension is as expected (or not expected). If expected and validated, the arterial dimensions and compliance curves may be used for blood pressure estimation. If not, recalibration (or calibration) or new measurements may be performed.

Finally, the compliance information and the zero-pressure arterial dimension may be used to determine a characteristic of the blood vessel (e.g., PWV) and with it, determine blood pressure. The following version of the Bramwell-Hill equation (Eqn. 1) provides a relationship between arterial distensibility, pressure variation, and PWV:

$$PWV = \sqrt{\frac{A}{ρ} \frac{dP}{dA}} \qquad ( Eqn . 1 )$$

A modification of Eqn. 1 yields:

$$dP = ρ {PWV}^{2} \frac{dA}{A} \qquad ( Eqn . 2 )$$

Assuming the PWV remains relatively constant during a cardiac cycle, integrating Eqn. 2 yields Eqn. 3 below:

$$P ( t ) = {P}_{0} + ρ {PWV}^{2} ln ( \frac{A ( t )}{{A}_{0}} ) \qquad ( Eqn . 3 )$$

P(t) can be further defined as follows, where Pᵢ(t) is the blood pressure to be determined, and Pₑ(t) is the known applied external pressure:

$$P ( t ) = {P}_{i} ( t ) - {P}_{e} ( t ) \qquad ( Eqn . 4 )$$

In the equations provided herein, A is the mean cross-sectional area at a given external pressure, which can be obtained using photoacoustic measurements as described above. dP/dA (or dP/dV) is the inverse of compliance. As described above with respect to FIG. 10, dP/dA (or dP/dV) can be obtained using a compliance curve, such as by identifying a slope (dA/dP) of curve 1000 at the given external pressure. ρ is blood density, which can be assumed to be a constant value for every user (although blood density may vary slightly for the individual and is not universally fixed). In some cases, ρ can be measured and determined for a user for greater personalization using means such as an oscillator for measuring fluid density.

Hence, compliance information can result in an estimation of PWV and blood pressure. The above approach can be useful during continuous blood pressure monitoring where obtaining photoacoustic measurements for dimensions and other characteristics (e.g., distension, compliance) can be used to estimate blood pressure thereby.

In some approaches (e.g., in the absence of a compliance curve), using a biometric sensor (e.g., photoacoustic sensor, acoustic sensor), PWV can be measured at external pressure Pₑ(t), and similarly, cross-sectional area A(t) can be measured. A₀ is an unknown cross-sectional area for Pₑ (0) and Pᵢ (0), where Pᵢ (0) is an unknown reference blood pressure, and Pₑ (0) is a known reference external pressure.

P₀ is a reference pressure of a difference between Pᵢ (0) and Pₑ (0):

$${P}_{0} = {P}_{i} ( 0 ) - {P}_{e} ( 0 ) \qquad ( Eqn . 5 )$$

Given the above, different external pressures can be applied at different times to determine blood pressure Pᵢ(t) and applied external pressures Pₑ(t). Combining Eqns. 3-5 yields:

$${P}_{i} ( t ) - {P}_{e} ( t ) = [ {P}_{i} ( 0 ) - {P}_{e} ( 0 ) ] + ρ {PWV}^{2} ln ( \frac{A ( t )}{{A}_{0}} ) \qquad ( Eqn . 6 )$$

Pᵢ(t), the blood pressure to be determined, can now be formulated as follows:

$${P}_{i} ( t ) = {P}_{i} ( 0 ) - {P}_{e} ( 0 ) + {P}_{e} ( t ) + ρ {PWV}^{2} ln ( \frac{A ( t )}{{A}_{0}} ) \qquad ( Eqn . 7 )$$

From here, information about multiple discrete pressure levels (e.g., three or more pressure levels) at different times can be used. For example, at t₁, t₂ and t₃, different respective cuff pressures Pₑ (t₁), Pₑ (t₂) and Pₑ (t₃) can be applied to the user to result in a set of equations:

$${P}_{i} ( {t}_{1} ) = {P}_{i} ( 0 ) - {P}_{e} ( 0 ) + {P}_{e} ( {t}_{1} ) + ρ {PWV ( {t}_{1} )}^{2} ln ( \frac{A ( {t}_{1} )}{{A}_{0}} ) \qquad ( Eqn . 8 a )$$

$${P}_{i} ( {t}_{2} ) = {P}_{i} ( 0 ) - {P}_{e} ( 0 ) + {P}_{e} ( {t}_{2} ) + ρ {PWV ( {t}_{2} )}^{2} ln ( \frac{A ( {t}_{2} )}{{A}_{0}} ) \qquad ( Eqn . 8 b )$$

$${P}_{i} ( {t}_{3} ) = {P}_{i} ( 0 ) - {P}_{e} ( 0 ) + {P}_{e} ( {t}_{3} ) + ρ {PWV ( {t}_{3} )}^{2} ln ( \frac{A ( {t}_{3} )}{{A}_{0}} ) \qquad ( Eqn . 8 c )$$

Three different external pressures are provided at three different times. Three unknowns include the blood pressure to be determined Pᵢ(t) (which is equal to Pᵢ (t₁), Pᵢ (t₂), and Pᵢ (t₃) since blood pressure of the user is assumed to remain relatively constant at t₁, t₂ and t₃), Pᵢ (0), and A₀, which can be solved using three Eqns. 8a-8c.

In another approach, a zero-pressure condition can be considered, where Pₑ(t)=0. In such a scenario, P(t) would equal Pᵢ(t), the blood pressure to be determined, according to Eqn. 4. The zero-pressure arterial dimension (area or volume) can be determined via extrapolation as shown with respect to FIG. 11. This information can be applied using the above approaches to estimate the blood pressure.

In yet another approach, other variables relating to the blood vessel can be used to determine the blood pressure. Consider the Shapiro equation:

$$\frac{{p}_{i} - {p}_{e}}{{κ}_{p}} ∝ {( \frac{A}{{A}_{0}} )}^{- n} \qquad ( Eqn . 9 )$$

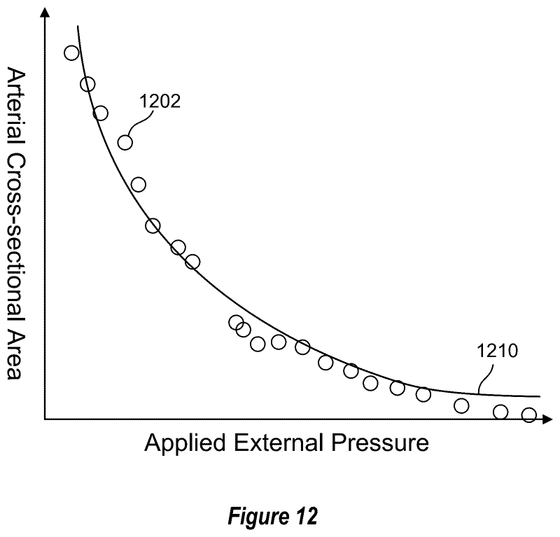

Here, pᵢ is blood pressure of the blood vessel, pₑ is external pressure applied to the blood vessel (e.g., via a cuff), κₚ is critical pressure of vessel collapse, A is current cross-sectional lumen area, A₀ is cross-sectional lumen area in the unstressed state, and n is a constant that depends on one or more factors that change from individual to individual to provide the fit shown in FIG. 12.

FIG. 12 is a graph of an example set of data points of arterial cross-sectional areas 1202 as a function of applied external pressure, according to some implementation. Arterial cross-sectional areas 1202 may be acquired from experimental data. An example range of applied external pressure may be 60-85 mmHg. An example range of arterial cross-sectional areas may be a normalized range from 0 to 1 (mm²/mm²). In some cases, a curve 1210 may be fitted on the data points of arterial cross-sectional areas 1202 using Eqn. 9. Thus, the blood pressure pᵢ, may be determined based on the applied external pressure and empirical measurements.

In some embodiments, a machine learning model may be used to predict a physiological parameter, e.g., blood pressure of the user. A machine learning model may refer to a computational algorithm that indicates relationships between input variables and output variables. In some embodiments, a machine learning model can be trained. Training a machine learning model may involve, among other things, determining values of weights associated with the machine learning model, where relationships between the input variables and the output variables are based at least in part on the determined weight values. In one implementation, a machine learning model may be trained in a supervised manner using a training set that includes labeled training data. In a more particular example, the labeled training data may include inputs and manually annotated outputs that the machine learning model is to approximate using determined weight values. In another implementation, a machine learning model may be trained in an unsupervised manner in which weight values are determined without manually labeled training data.

An example training process for the machine learning model may involve providing training data that includes known photoacoustic signal data, known arterial spatial measurements (e.g., area, volume), known external pressure levels, known associated compliance information, and/or known zero-condition arterial dimensions, as well as the “ground truth” or the known output characteristics or parameters, e.g., PWV, blood pressure, or cardiac features (e.g., peaks or HRW features) that are known. In some approaches, a portion (e.g., 20%) of the training data may be used as part of a validation set for the machine learning model. With this training data and validation set, one or more loss functions may be implemented. A loss function is an optimization function in which an error is iteratively minimized through, e.g., gradient descent. A productive learning rate that dictates the “step” the gradient descent takes when finding the lowest error may be set during training as well.

As a result, a trained machine learning model can be generated. In some implementations, such a trained machine learning model can be used to further enhance the accuracy and reliability of the estimated physiological characteristics or parameter. For example, an estimation derived from measurements from the disclosed sensor apparatus and various derivations based on photoacoustic measurements can be provided to the machine learning model (stored at a sensor or a host device and/or accessible by a control system thereof) to compare with a physiological characteristic of a blood vessel (e.g., PTT, PWV, heart rate) or a physiological parameter of a user (e.g., blood pressure) estimated by the machine learning model. If there is a discrepancy between the sensor-based estimation and the model-generated prediction which is greater than a threshold, the obtained estimation may be further evaluated or discarded. If discarded, the model-generated prediction may be used, or additional measurements may be taken by the sensors. In cases where there are more than two sensors on the user and a discrepancy arises between the sensor estimations and the model predictions, fewer sensors may be used as a fallback rather than all sensors. On the other hand, if the discrepancy is lower than a threshold, the sensor-based estimation may be selected or kept for further processing, sending to a host device, reporting, displaying to the user, etc.

### Example Methods

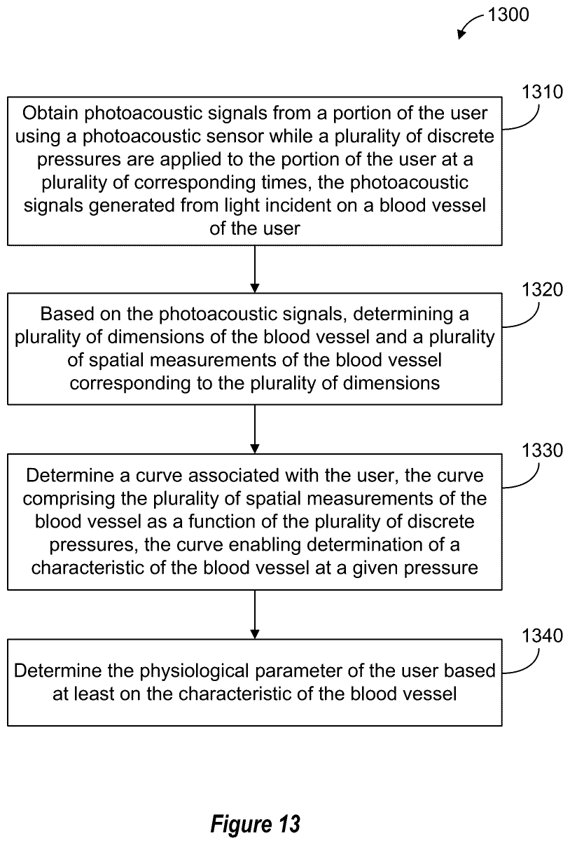

FIG. 13 is a flow diagram of a method 1300 for determining a physiological parameter of a user, according to some disclosed implementations. Structure for performing the functionality illustrated in one or more of the blocks shown in FIG. 13 may be performed by hardware and/or software components of a computerized apparatus or system (which may be implemented as a wearable device in some embodiments). Components of such apparatus or system may include, for example, one or more sensors, a control system (including one or more processors), a memory, and/or a computer-readable apparatus including a storage medium storing computer-readable and/or computer-executable instructions that are configured to, when executed by the control system, cause the control system, the one or more processors, or the apparatus to perform operations represented by blocks below. Example components of the apparatus are illustrated in FIG. 2, which are described in more detail above.

The blocks of FIG. 13 may, for example, be performed by the apparatus 200 or by a similar apparatus, or a component thereof (e.g., a control system). As with other methods disclosed herein, the method outlined in FIG. 13 may include more or fewer blocks than indicated. Moreover, the blocks of methods disclosed herein are not necessarily performed in the order indicated. In some instances, one or more of the blocks shown in FIG. 13 may be performed concurrently.

At block 1310, the method 1300 may include obtaining photoacoustic signals from a portion of a user using a photoacoustic sensor while a plurality of discrete pressures are applied to the portion of the user at a plurality of corresponding times, the photoacoustic signals generated from light incident on a blood vessel of the user. In some embodiments, the plurality of discrete pressures may be applied to the portion of the user, the portion of the user comprising skin of the user, using a cuff of a wearable device, the wearable device comprising the photoacoustic sensor and the cuff.

In some embodiments, the photoacoustic sensor may be configured to interface with the portion of the user, the portion of the user including skin of the user. In some embodiments, the wearable device may include a wearable structure, and the wearable structure may be configured to be worn around an appendage of the user. The appendage may be, e.g., wrist, finger, ankle, or other compatible body parts.

Means for performing functionality at block 1310 may include, the interface 201, the receiver system 202, and the light source system 204, the cuff system 205 and/or other components of the apparatus as shown in FIG. 2.

At block 1320, the method 1300 may include, based on the photoacoustic signals, determining a plurality of dimensions of the blood vessel and a plurality of spatial measurements of the blood vessel corresponding to the plurality of dimensions. In some embodiments, the plurality of dimensions may include a plurality of diameters of the blood vessel or a plurality of semi-axes of the blood vessel; and the method may further include deriving the plurality of spatial measurements from the diameters of the blood vessel or the plurality of semi-axes of the blood vessel, the plurality of spatial measurements including a plurality of cross-sectional areas associated with the blood vessel or a plurality of volumes associated with the blood vessel, and correlating to the characteristic of the blood vessel. Examples of semi-axes are a semi-major axis and a semi-minor axis. In some cases (e.g., where the volume of an ellipsoid blood vessel is determined), a third semi-axis is another example of a semi-axis.

In some embodiments, the method 1300 may include determining the plurality of dimensions using an image processing algorithm applied to an image representation of the photoacoustic signals, a machine learning model trained to obtain the first axis and the second axis based on the photoacoustic signals, or a combination thereof. In some cases, the plurality of spatial measurements of the blood vessel may be determined based on the plurality of dimensions, the first axis, the second axis, or a combination thereof.

Means for performing functionality at block 1320 may include, the control system 206 and/or other components of the apparatus as shown in FIG. 2.

At block 1330, the method 1300 may include determining a curve associated with the user, the curve comprising the plurality of spatial measurements of the blood vessel as a function of the plurality of discrete pressures, the curve enabling determination of a characteristic of the blood vessel at a given pressure. In some embodiments, the characteristic of the blood vessel may include a compliance of the blood vessel.

In some embodiments, the compliance of the blood vessel may be obtainable from a compliance curve associated with the user, the compliance curve determined based on a calibration process. In some implementations, the compliance curve may include a plurality of spatial parameters of the blood vessel as a function of a plurality of discrete pressures, the plurality of spatial parameters determined based on a plurality of dimensions of the blood vessel determined from the photoacoustic signals obtained while the plurality of discrete pressures are applied to the portion of the user at a plurality of corresponding times. In some implementations, the calibration process of the wearable user device may include obtaining the plurality of spatial parameters of the blood vessel corresponding to respective ones of a plurality of discrete pressures applied to the portion of the user by the cuff at a plurality of corresponding times, the plurality of spatial parameters associated with the blood vessel correlated to a plurality of dimensions of the blood vessel determined from the photoacoustic signals, the compliance curve comprising the plurality of spatial parameters as a function of the plurality of discrete pressures. Examples of the plurality of discrete pressures may range from 30-100 mmHg. In some cases, a pressure applied to the portion of the user may be a pressure of about 40 mmHg or less. In some cases, the pressure applied to the portion of the user may be a pressure of about 90 mmHg or less. However, the plurality of discrete pressures may be other pressure values, e.g., above 0 but below 30 mmHg, or above 100 mmHg up to a point of discomfort to the user. In some approaches, the plurality of spatial parameters of the blood vessel may include a plurality of cross-sectional areas associated with the blood vessel at the respective ones of the plurality of discrete pressures applied to the portion of the user by the cuff at corresponding times. In some other approaches, the plurality of spatial parameters of the blood vessel may include a plurality of arterial volumes associated with the blood vessel at the respective ones of the plurality of discrete pressures applied to the portion of the user by the cuff at corresponding times. In some cases, the plurality of spatial parameters of the blood vessel may include a plurality of cross-sectional areas associated with the blood vessel at the respective ones of the plurality of discrete pressures applied to the portion of the user by the cuff at corresponding times. In some implementations, a pulse wave velocity (PWV) of the blood vessel may correlate to the compliance of the blood vessel, the PWV being otherwise obtainable using the photoacoustic sensor and a second photoacoustic sensor that is not used by the wearable user device. In some implementations, a slope of the compliance curve may correspond to a compliance at the pressure applied to the portion of the user by the cuff.

In some embodiments, dimensions of the blood vessel may include a first axis of the blood vessel and a second axis of the blood vessel; one or more spatial parameters of the blood vessel may be derived from at least on the first axis and the second axis; and the one or more spatial parameters of the blood vessel and the pressure may correlate to the characteristic of the blood vessel. Examples of spatial parameters may be volume or cross-sectional area associated with the blood vessel. In some implementations, the first axis and the second axis may be determined using an image processing algorithm applied to an image representation of the photoacoustic signals, a machine learning model trained to obtain the first axis and the second axis based on the photoacoustic signals, or a combination thereof.

Means for performing functionality at block 1330 may include, the control system 206 and/or other components of the apparatus as shown in FIG. 2.

At block 1340, the method 1300 may include determining the physiological parameter of the user based at least on the characteristic of the blood vessel. In some embodiments, the physiological parameter of the user may include a blood pressure of the user. In some implementations, the determining of the physiological parameter of the user may include determining a blood pressure of the user based at least on the characteristic; wherein the characteristic may include a compliance of the blood vessel; the method may further include determining a pulse wave velocity (PWV) of the blood vessel based at least on the compliance; and the determining of the blood pressure of the user may include determining the blood pressure of the user based at least on the PWV.

Means for performing functionality at block 1340 may include, the control system 206 and/or other components of the apparatus as shown in FIG. 2.

In some embodiments, the characteristic of the blood vessel may further enable determination of a pulse wave velocity associated with the blood vessel. In some implementations, the blood pressure of the user may be determined. In some implementations, the blood pressure of the user may be determined based at least on an estimated parameter of the blood vessel, the estimated parameter of the blood vessel comprising a diameter associated with the blood vessel at zero pressure, and determined via (i) extrapolation of a plurality of diameters as a function of a plurality of discrete pressures applied to the portion of the user by the cuff, or (ii) a machine learning model trained to determine the diameter based on the plurality of discrete pressures.

In some embodiments, the cuff may be further configured to apply the pressure to the portion of the user at a plurality of substantially constant pressure levels, the plurality of substantially constant pressure levels being different from one another; and the blood pressure of the user may be determined based at least on the plurality of substantially constant pressure levels. Eqns. 8a-8c may be used, for instance.

In some embodiments, the wearable device may further include a control system, wherein the control system may be configured to determine the characteristic of the blood vessel and the blood pressure of the user based at least on the characteristic of the blood vessel.

In some embodiments, the method 1300 may further include determining a pulse wave velocity (PWV) of the blood vessel based at least on the compliance of the blood vessel, the PWV being otherwise obtainable using the photoacoustic sensor and a second photoacoustic sensor that is not used.

In some embodiments, the method 1300 may further include: subsequent to the determining of the curve associated with the user, applying a pressure to the portion of the user at a substantially constant pressure level; and determining, based on a slope of the curve, the characteristic of the blood vessel corresponding to the substantially constant pressure level.

In some embodiments, the method 1300 may further include, subsequent to the determining of the curve, applying the pressure to the portion of the user at a plurality of substantially constant pressure levels, the plurality of substantially constant pressure levels being different from one another. In some implementations, the determining of the physiological parameter of the user may be further based at least on the plurality of substantially constant pressure levels.

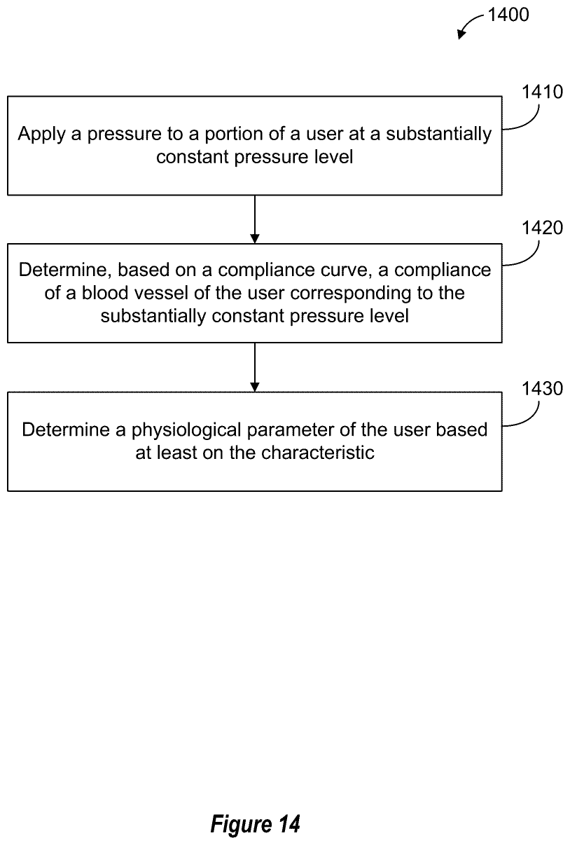

FIG. 14 is a flow diagram of a method 1400 for determining a physiological parameter of a user, according to some disclosed implementations. Structure for performing the functionality illustrated in one or more of the blocks shown in FIG. 14 may be performed by hardware and/or software components of a computerized apparatus or system (which may be implemented as a wearable device in some embodiments). Components of such apparatus or system may include, for example, one or more sensors, a control system (including one or more processors), a memory, and/or a computer-readable apparatus including a storage medium storing computer-readable and/or computer-executable instructions that are configured to, when executed by the control system, cause the control system, the one or more processors, or the apparatus to perform operations represented by blocks below. Example components of the apparatus are illustrated in FIG. 2, which are described in more detail above.

The blocks of FIG. 14 may, for example, be performed by the apparatus 200 or by a similar apparatus, or a component thereof (e.g., a control system). As with other methods disclosed herein, the method outlined in FIG. 14 may include more or fewer blocks than indicated. Moreover, the blocks of methods disclosed herein are not necessarily performed in the order indicated. In some instances, one or more of the blocks shown in FIG. 14 may be performed concurrently.

At block 1410, the method 1400 may include applying a pressure to a portion of a user at a substantially constant pressure level. As noted above, examples of the substantially constant pressure level may include about 30 mmHg, about 100 mmHg, or less.

Means for performing functionality at block 1410 may include, the cuff system 205 and/or other components of the apparatus as shown in FIG. 2.

At block 1420, the method 1400 may include determining, based on a compliance curve, a compliance of a blood vessel of the user corresponding to the substantially constant pressure level. For instance, a slope at the applied external pressure can be determined from curve 1000 shown in FIG. 10, where the slope corresponds to the compliance of the blood vessel.

Means for performing functionality at block 1420 may include, the control system 206 and/or other components of the apparatus as shown in FIG. 2.

At block 1430, the method 1400 may include determining a physiological parameter of the user based at least on the characteristic. In some embodiments, the physiological parameter of the user may include a blood pressure.

Means for performing functionality at block 1430 may include, the control system 206 and/or other components of the apparatus as shown in FIG. 2.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

Implementation examples are described in the following numbered clauses:

Clause 1: A wearable user device comprising: a cuff configured to apply a pressure to a portion of a user at one or more substantially constant pressure levels; and a photoacoustic sensor configured to obtain photoacoustic signals generated from light incident on a blood vessel of the user, the photoacoustic signals correlated to one or more dimensions of the blood vessel of the user while the pressure is applied to the portion of the user, the one or more dimensions of the blood vessel and the pressure correlating to a characteristic of the blood vessel, the characteristic of the blood vessel enabling determination of a blood pressure of the user; and a wearable structure comprising the cuff and the photoacoustic sensor.

Clause 2: The wearable user device of clause 1, wherein: the characteristic of the blood vessel comprises a compliance of the blood vessel; and the compliance of the blood vessel is obtainable from a compliance curve associated with the user, the compliance curve determined based on a calibration process.

Clause 3: The wearable user device of any one of clauses 1-2 wherein the compliance curve comprises a plurality of spatial parameters of the blood vessel as a function of a plurality of discrete pressures, the plurality of spatial parameters determined based on a plurality of dimensions of the blood vessel determined from the photoacoustic signals obtained while the plurality of discrete pressures are applied to the portion of the user at a plurality of corresponding times.

Clause 4: The wearable user device of any one of clauses 1-3 wherein the calibration process of the wearable user device comprises obtaining the plurality of spatial parameters of the blood vessel corresponding to respective ones of a plurality of discrete pressures applied to the portion of the user by the cuff at a plurality of corresponding times, the plurality of spatial parameters associated with the blood vessel correlated to a plurality of dimensions of the blood vessel determined from the photoacoustic signals, the compliance curve comprising the plurality of spatial parameters as a function of the plurality of discrete pressures.

Clause 5: The wearable user device of any one of clauses 1-4 wherein the plurality of spatial parameters of the blood vessel comprise a plurality of cross-sectional areas associated with the blood vessel at the respective ones of the plurality of discrete pressures applied to the portion of the user by the cuff at corresponding times.

Clause 6: The wearable user device of any one of clauses 1-5 wherein a pulse wave velocity (PWV) of the blood vessel correlates to the compliance of the blood vessel, the PWV being otherwise obtainable using the photoacoustic sensor and a second photoacoustic sensor that is not used by the wearable user device.

Clause 7: The wearable user device of any one of clauses 1-6 wherein a slope of the compliance curve corresponds to a compliance at the pressure applied to the portion of the user by the cuff.

Clause 8: The wearable user device of any one of clauses 1-7 wherein the one or more dimensions of the blood vessel comprise a first axis of the blood vessel and a second axis of the blood vessel; one or more spatial parameters of the blood vessel are derived from at least on the first axis and the second axis; and the one or more spatial parameters of the blood vessel and the pressure correlate to the characteristic of the blood vessel.

Clause 9: The wearable user device of any one of clauses 1-8 wherein the first axis and the second axis are determined using an image processing algorithm applied to an image representation of the photoacoustic signals, a machine learning model trained to obtain the first axis and the second axis based on the photoacoustic signals, or a combination thereof.

Clause 10: The wearable user device of any one of clauses 1-9 wherein the characteristic of the blood vessel further enables determination of a pulse wave velocity associated with the blood vessel.

Clause 11: The wearable user device of any one of clauses 1-10 wherein the blood pressure of the user is determined based at least on the pulse wave velocity associated with the blood vessel.

Clause 12: The wearable user device of any one of clauses 1-11 wherein the blood pressure of the user is determined based at least on an estimated parameter of the blood vessel, the estimated parameter of the blood vessel comprising a diameter associated with the blood vessel at zero pressure, and determined via (i) extrapolation of a plurality of diameters as a function of a plurality of discrete pressures applied to the portion of the user by the cuff, or (ii) a machine learning model trained to determine the diameter based on the plurality of discrete pressures.

Clause 13: The wearable user device of any one of clauses 1-12 wherein the characteristic of the blood vessel comprises a pulse wave velocity of the blood vessel determined using the photoacoustic signals; the cuff is further configured to apply the pressure to the portion of the user at a plurality of substantially constant pressure levels, the plurality of substantially constant pressure levels being different from one another; and the blood pressure of the user is determined based at least on the pulse wave velocity and the plurality of substantially constant pressure levels.

Clause 14: The wearable user device of any one of clauses 1-13 wherein the photoacoustic sensor is configured to interface with the portion of the user, the portion of the user comprising skin of the user.

Clause 15: The wearable user device of any one of clauses 1-14 wherein the wearable structure is configured to be worn around an appendage of the user.

Clause 16: The wearable user device of any one of clauses 1-15 wherein the pressure applied to the portion of the user comprises a pressure of about 40 mmHg or less.

Clause 17: The wearable user device of any one of clauses 1-16 wherein the pressure applied to the portion of the user comprises a pressure of about 90 mmHg or less.

Clause 18: The wearable user device of any one of clauses 1-17 further comprising a control system, wherein the control system is configured to determine the characteristic of the blood vessel and the blood pressure of the user based at least on the characteristic of the blood vessel.

Clause 19: A method of determining a physiological parameter of a user, the method comprising: obtaining photoacoustic signals from a portion of the user using a photoacoustic sensor while a plurality of discrete pressures are applied to the portion of the user at a plurality of corresponding times, the photoacoustic signals generated from light incident on a blood vessel of the user; based on the photoacoustic signals, determining a plurality of dimensions of the blood vessel and a plurality of spatial measurements of the blood vessel corresponding to the plurality of dimensions; determining a curve associated with the user, the curve comprising the plurality of spatial measurements of the blood vessel as a function of the plurality of discrete pressures, the curve enabling determination of a characteristic of the blood vessel at a given pressure; and determining the physiological parameter of the user based at least on the characteristic of the blood vessel.

Clause 20: The method of clause 19, wherein the physiological parameter of the user comprises a blood pressure of the user.

Clause 21: The method of any one of clauses 19-20 wherein the characteristic of the blood vessel comprises a compliance of the blood vessel.

Clause 22: The method of any one of clauses 19-21 further comprising determining a pulse wave velocity (PWV) of the blood vessel based at least on the compliance of the blood vessel, the PWV being otherwise obtainable using the photoacoustic sensor and a second photoacoustic sensor that is not used.

Clause 23: The method of any one of clauses 19-22 further comprising subsequent to the determining of the curve associated with the user, applying a pressure to the portion of the user at a substantially constant pressure level; and determining, based on a slope of the curve, the characteristic of the blood vessel corresponding to the substantially constant pressure level; wherein the determining of the physiological parameter of the user comprises determining a blood pressure of the user based at least on the characteristic.

Clause 24: The method of any one of clauses 19-23 wherein the characteristic comprises a compliance of the blood vessel; the method further comprises determining a pulse wave velocity (PWV) of the blood vessel based at least on the compliance; and the determining of the blood pressure of the user comprises determining the blood pressure of the user based at least on the PWV.

Clause 25: The method of any one of clauses 19-24 wherein the plurality of dimensions comprise a plurality of diameters of the blood vessel or a plurality of semi-axes of the blood vessel; and the method further comprises deriving the plurality of spatial measurements from the diameters of the blood vessel or the plurality of semi-axes of the blood vessel, the plurality of spatial measurements comprising a plurality of cross-sectional areas associated with the blood vessel or a plurality of volumes associated with the blood vessel, and correlating to the characteristic of the blood vessel.

Clause 26: The method of any one of clauses 19-25 further comprising determining the plurality of dimensions using an image processing algorithm applied to an image representation of the photoacoustic signals, a machine learning model trained to obtain the first axis and the second axis based on the photoacoustic signals, or a combination thereof.

Clause 27: The method of any one of clauses 19-26 wherein the plurality of discrete pressures are applied to the portion of the user, the portion of the user comprising skin of the user, using a cuff of a wearable device, the wearable device comprising the photoacoustic sensor and the cuff.

Clause 28: The method of any one of clauses 19-27 further comprising subsequent to the determining of the curve, applying the pressure to the portion of the user at a plurality of substantially constant pressure levels, the plurality of substantially constant pressure levels being different from one another; wherein the determining of the physiological parameter of the user is further based at least on the plurality of substantially constant pressure levels.

Clause 29: An apparatus comprising: means for applying a pressure to a portion of a user at a substantially constant pressure level; means for obtaining photoacoustic signals generated from light incident on a blood vessel of the user, the photoacoustic signals correlated to one or more dimensions of the blood vessel of the user while the pressure is applied to the portion of the user, the one or more dimensions of the blood vessel and the pressure correlating to a characteristic of the blood vessel, the characteristic of the blood vessel enabling determination of a blood pressure of the user; and wearable means comprising the means for applying the pressure to the portion of the user and the means for obtaining the photoacoustic signals.

Clause 30: A non-transitory computer-readable apparatus comprising a storage medium, the storage medium comprising a plurality of instructions configured to, when executed by one or more processors, cause an apparatus to: obtain photoacoustic signals from a portion of the user using a photoacoustic sensor while a plurality of discrete pressures are applied to the portion of the user at a plurality of corresponding times, the photoacoustic signals generated from light incident on a blood vessel of the user; based on the photoacoustic signals, determine a plurality of dimensions of the blood vessel and a plurality of spatial measurements of the blood vessel corresponding to the plurality of dimensions; determine a curve associated with the user, the curve comprising the plurality of spatial measurements of the blood vessel as a function of the plurality of discrete pressures, the curve enabling determination of a characteristic of the blood vessel at a given pressure; and determine the physiological parameter of the user based at least on the characteristic of the blood vessel.

What is claimed:

1. A wearable user device comprising:
a cuff configured to apply a pressure to a portion of a user at one or more substantially constant pressure levels; and
a photoacoustic sensor configured to obtain photoacoustic signals generated from light incident on a blood vessel of the user, the photoacoustic signals correlated to one or more dimensions of the blood vessel of the user while the pressure is applied to the portion of the user, the one or more dimensions of the blood vessel and the pressure correlating to a characteristic of the blood vessel, the characteristic of the blood vessel enabling determination of a blood pressure of the user; and
a wearable structure comprising the cuff and the photoacoustic sensor.

2. The wearable user device of claim 1, wherein:
the characteristic of the blood vessel comprises a compliance of the blood vessel; and
the compliance of the blood vessel is obtainable from a compliance curve associated with the user, the compliance curve determined based on a calibration process.

3. The wearable user device of claim 2, wherein the compliance curve comprises a plurality of spatial parameters of the blood vessel as a function of a plurality of discrete pressures, the plurality of spatial parameters determined based on a plurality of dimensions of the blood vessel determined from the photoacoustic signals obtained while the plurality of discrete pressures are applied to the portion of the user at a plurality of corresponding times.

4. The wearable user device of claim 2, wherein the calibration process of the wearable user device comprises obtaining a plurality of spatial parameters of the blood vessel corresponding to respective ones of a plurality of discrete pressures applied to the portion of the user by the cuff at a plurality of corresponding times, the plurality of spatial parameters associated with the blood vessel correlated to a plurality of dimensions of the blood vessel determined from the photoacoustic signals, the compliance curve comprising the plurality of spatial parameters as a function of the plurality of discrete pressures.

5. The wearable user device of claim 4, wherein the plurality of spatial parameters of the blood vessel comprise a plurality of cross-sectional areas associated with the blood vessel at the respective ones of the plurality of discrete pressures applied to the portion of the user by the cuff at corresponding times.

6. The wearable user device of claim 2, wherein a pulse wave velocity (PWV) of the blood vessel correlates to the compliance of the blood vessel, the PWV being otherwise obtainable using the photoacoustic sensor and a second photoacoustic sensor that is not used by the wearable user device.

7. The wearable user device of claim 2, wherein a slope of the compliance curve corresponds to a compliance at the pressure applied to the portion of the user by the cuff.

8. The wearable user device of claim 1, wherein:
the one or more dimensions of the blood vessel comprise a first axis of the blood vessel and a second axis of the blood vessel;
one or more spatial parameters of the blood vessel are derived from at least on the first axis and the second axis; and
the one or more spatial parameters of the blood vessel and the pressure correlate to the characteristic of the blood vessel.

9. The wearable user device of claim 8, wherein the first axis and the second axis are determined using an image processing algorithm applied to an image representation of the photoacoustic signals, a machine learning model trained to obtain the first axis and the second axis based on the photoacoustic signals, or a combination thereof.

10. The wearable user device of claim 1, wherein the characteristic of the blood vessel further enables determination of a pulse wave velocity associated with the blood vessel.

11. The wearable user device of claim 10, wherein the blood pressure of the user is determined based at least on the pulse wave velocity associated with the blood vessel.

12. The wearable user device of claim 10, wherein the blood pressure of the user is determined based at least on an estimated parameter of the blood vessel, the estimated parameter of the blood vessel comprising a diameter associated with the blood vessel at zero pressure, and determined via (i) extrapolation of a plurality of diameters as a function of a plurality of discrete pressures applied to the portion of the user by the cuff, or (ii) a machine learning model trained to determine the diameter based on the plurality of discrete pressures.

13. The wearable user device of claim 1, wherein:
the characteristic of the blood vessel comprises a pulse wave velocity of the blood vessel determined using the photoacoustic signals;
the cuff is further configured to apply the pressure to the portion of the user at a plurality of substantially constant pressure levels, the plurality of substantially constant pressure levels being different from one another; and
the blood pressure of the user is determined based at least on the pulse wave velocity and the plurality of substantially constant pressure levels.

14. The wearable user device of claim 1, wherein the photoacoustic sensor is configured to interface with the portion of the user, the portion of the user comprising skin of the user.

15. The wearable user device of claim 1, wherein the wearable structure is configured to be worn around an appendage of the user.

16. The wearable user device of claim 1, wherein the pressure applied to the portion of the user comprises a pressure of about 40 mmHg or less.

17. The wearable user device of claim 1, wherein the pressure applied to the portion of the user comprises a pressure of about 90 mmHg or less.

18. The wearable user device of claim 1, further comprising a control system, wherein the control system is configured to determine the characteristic of the blood vessel and the blood pressure of the user based at least on the characteristic of the blood vessel.

19. A method of determining a physiological parameter of a user, the method comprising:
obtaining photoacoustic signals from a portion of the user using a photoacoustic sensor while a plurality of discrete pressures are applied to the portion of the user at a plurality of corresponding times, the photoacoustic signals generated from light incident on a blood vessel of the user;
based on the photoacoustic signals, determining a plurality of dimensions of the blood vessel and a plurality of spatial measurements of the blood vessel corresponding to the plurality of dimensions;
determining a curve associated with the user, the curve comprising the plurality of spatial measurements of the blood vessel as a function of the plurality of discrete pressures, the curve enabling determination of a characteristic of the blood vessel at a given pressure; and
determining the physiological parameter of the user based at least on the characteristic of the blood vessel.

20. The method of claim 19, wherein the physiological parameter of the user comprises a blood pressure of the user.

21. The method of claim 19, wherein the characteristic of the blood vessel comprises a compliance of the blood vessel.

22. The method of claim 21, further comprising determining a pulse wave velocity (PWV) of the blood vessel based at least on the compliance of the blood vessel, the PWV being otherwise obtainable using the photoacoustic sensor and a second photoacoustic sensor that is not used.

23. The method of claim 19, further comprising:
subsequent to the determining of the curve associated with the user, applying a pressure to the portion of the user at a substantially constant pressure level; and
determining, based on a slope of the curve, the characteristic of the blood vessel corresponding to the substantially constant pressure level;
wherein the determining of the physiological parameter of the user comprises determining a blood pressure of the user based at least on the characteristic.

24. The method of claim 23, wherein:
the characteristic comprises a compliance of the blood vessel;
the method further comprises determining a pulse wave velocity (PWV) of the blood vessel based at least on the compliance; and
the determining of the blood pressure of the user comprises determining the blood pressure of the user based at least on the PWV.

25. The method of claim 19, wherein:
the plurality of dimensions comprise a plurality of diameters of the blood vessel or a plurality of semi-axes of the blood vessel; and
the method further comprises deriving the plurality of spatial measurements from the diameters of the blood vessel or the plurality of semi-axes of the blood vessel, the plurality of spatial measurements comprising a plurality of cross-sectional areas associated with the blood vessel or a plurality of volumes associated with the blood vessel, and correlating to the characteristic of the blood vessel.

26. The method of claim 19, further comprising determining the plurality of dimensions using an image processing algorithm applied to an image representation of the photoacoustic signals, a machine learning model trained to obtain a first axis and a second axis of the blood vessel based on the photoacoustic signals, or a combination thereof;
wherein the plurality of spatial measurements of the blood vessel are determined based on the plurality of dimensions, the first axis, the second axis, or a combination thereof.

27. The method of claim 19, wherein the plurality of discrete pressures are applied to the portion of the user, the portion of the user comprising skin of the user, using a cuff of a wearable device, the wearable device comprising the photoacoustic sensor and the cuff.

28. The method of claim 19, wherein the plurality of discrete pressures range from about 30 to about 100 mmHg.

29. An apparatus comprising:
means for applying a pressure to a portion of a user at a substantially constant pressure level;
means for obtaining photoacoustic signals generated from light incident on a blood vessel of the user, the photoacoustic signals correlated to one or more dimensions of the blood vessel of the user while the pressure is applied to the portion of the user, the one or more dimensions of the blood vessel and the pressure correlating to a characteristic of the blood vessel, the characteristic of the blood vessel enabling determination of a blood pressure of the user; and
wearable means comprising the means for applying the pressure to the portion of the user and the means for obtaining the photoacoustic signals.

30. A non-transitory computer-readable apparatus comprising a storage medium, the storage medium comprising a plurality of instructions configured to, when executed by one or more processors, cause an apparatus to:
obtain photoacoustic signals from a portion of a user using a photoacoustic sensor while a plurality of discrete pressures are applied to the portion of the user at a plurality of corresponding times, the photoacoustic signals generated from light incident on a blood vessel of the user;
based on the photoacoustic signals, determine a plurality of dimensions of the blood vessel and a plurality of spatial measurements of the blood vessel corresponding to the plurality of dimensions;
determine a curve associated with the user, the curve comprising the plurality of spatial measurements of the blood vessel as a function of the plurality of discrete pressures, the curve enabling determination of a characteristic of the blood vessel at a given pressure; and
determine a physiological parameter of the user based at least on the characteristic of the blood vessel.
